(19) United States
(12) Patent Application Publication
(10) Pub. No.: US 2022/0372435 A1
(43) Pub. Date: Nov. 24, 2022

(54) Cell Culture Media Tablets and Methods of Manufacture

(71) Applicant: LIFE TECHNOLOGIES CORPORATION, Carlsbad, CA (US)

(72) Inventor: Mwita PHELPS, Buffalo, NY (US)

(21) Appl. No.: 17/761,879

(22) PCT Filed: Sep. 17, 2020
(86) PCT No.: PCT/US20/51236

§ 371 Date: Mar. 18, 2022

Related U.S. Application Data
(60) Provisional application No. 62/902,703, filed on Sep. 19, 2019.

(51) Int. Cl. C12N 5/00

(52) U.S. Cl. CPC C12N 5/0018; C12N 2500/16; C12N 2500/32; C12N 2500/38; C12N 2500/36; C12N 2500/40; C12N 2500/34

(57) ABSTRACT

Described herein are efficiently dissolving tablets of dry cell culture media, feeds, supplements, media subgroups, buffer concentrates, or media components useful in culturing cells or microorganisms, methods of manufacturing, and methods of use. In particular, formulations of tableted cell culture media feeds, supplements, media subgroups, or buffer concentrates are described.

### CROSS REFERENCE TO RELATED APPLICATIONS

This application claims the benefit of priority under 35 U.S.C. § 119(e) of U.S. Provisional Application No. 62/902,703, filed on Sep. 19, 2019, which is incorporated by reference herein in its entirety.

### TECHNICAL FIELD

Described herein are efficiently dissolving tablets of dry cell culture media, feeds, supplements, media subgroups, buffer concentrates, or media components useful in culturing cells or microorganisms, methods of manufacturing, and methods of use. In particular, formulations of tableted cell culture media feeds, supplements, media subgroups, or buffer concentrates are described.

### BACKGROUND

Cell culture media provide the nutrients for maintaining or growing cells in a controlled in vitro environment. The characteristics and compositions of the cell culture media vary depending on the particular cellular requirements and any functions for which the cells are cultured. Media is typically manufactured as dry powders, liquids, liquid concentrates, agglomerated media, or agglomerated media pellets. See e.g., U.S. Pat. Nos. 6,383,810 and 6,627,426 and U.S. Pat. App. Pub. Nos. US 2018/0142203 A1 and US 20190048312 A1, each of which are incorporated by reference herein for teachings related to cell culture media. Each of these media formats has particular advantages and disadvantages. For example, dry powders are easy to use and store, but create potentially hazardous dusts and some components may be difficult to dissolve. Liquid media and liquid media concentrates are “ready to use,” but often require supplements, have short shelf lives, and are difficult to sterilize in bulk. Agglomerated media and pelleted media overcame many of the disadvantages of dry powder media. These media formats are dissolved and sterilized by the end user when needed and can be stored for up to 2 years. The only drawbacks to agglomerated and pelleted media is that they must be weighed to achieve uniform masses for targeted volumes and scale-up, and their manufacturing production rates are variable.

Thus, there is a need for tableted, dry, stable, efficiently dissolving, cell culture media products that can be mass-produced in bulk quantities with reduced processing steps, and that are amenable to typical sterilization methods, targeted volumes without weighing, and rapid scale-up.

### SUMMARY

One embodiment described herein is a tableted composition comprising a cell culture medium, feed, or supplement comprising: amino acids, salts, buffers, trace minerals, vitamins, carbohydrates, lipids, nucleic acids, proteins; and a lubricant, filler, binder, or combination thereof. In one aspect, the tableted composition dissolves within about 10-30 minutes in water at 25° C. In another aspect, the composition comprises: 95-99 percent by mass of amino acids, salts, buffers, trace minerals, vitamins, carbohydrates, lipids, nucleic acids, proteins; and 1-5 percent by mass of a lubricant comprising magnesium stearate. In another aspect, the composition comprises: 90-99 percent by mass of amino acids, salts, buffers, trace minerals, vitamins, carbohydrates, lipids, nucleic acids, proteins; and 1-5 percent by mass of a disintegrant comprising croscarmellose sodium; and 1-5 percent by mass of a lubricant comprising magnesium stearate.

Another embodiment described herein is a composition comprising a tableted cell culture medium, feed, or supplement composition comprising: a media component comprising: 20-65 percent by mass carbohydrates; 20-40 percent by mass amino acids; 2-10 percent by mass inorganic salts or buffers; 1-5 percent by mass vitamins; 0.01-0.05 percent by mass trace minerals; and a tableting component comprising: 1-10 percent by mass of a lubricant, filler, binder, or combinations thereof. In one aspect, the composition comprises: (a) 20-65 percent by mass of mono or disaccharides comprising glucose, fructose, lactose, trehalose, maltose, or sucrose; (b) 20-40 percent by mass of amino acids comprising alanine, arginine, aspartic acid, glutamic acid, glycine, histidine, isoleucine, methionine, phenylalanine, proline, hydroxyproline, serine, threonine, tryptophan, valine, tyrosine, cysteine, lysine, salts thereof, or combinations thereof; (c) 2-10 percent by mass of salts of sodium, potassium, magnesium, calcium, ammonium, phosphate, carbonate, sulfate, or combinations thereof; (d) 1-5 percent by mass of vitamins comprising retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, para-aminobenzoic acid, salts thereof, or combinations thereof; (e) 0.01-0.05 percent by mass of trace elements including iron, manganese, copper, iodine, zinc, cobalt, fluoride, chromium, molybdenum, selenium, nickel, silicon, vanadium, salts thereof, or combinations thereof; (f) 1-5 percent by mass of magnesium stearate; and (g) 1-5 percent by mass of croscarmellose sodium.

In another aspect, the media component comprises an agglomerated dry powder. In another aspect, the tableted medium dissolves within about 10-30 minutes in water at 25° C. In another aspect, the tableted medium has a hardness of about 18-22 kp. In another aspect, the tableted medium has a mass of about 2.0 to 5.0 g. In another aspect, the tableted medium upon reconstitution with water exhibits cell viability levels and expressed protein levels commensurate with an analogous non-tableted medium.

Another embodiment described herein is a method for producing a tableted cell culture medium, feed, or supplement composition, the method comprising: (a) preparing a cell culture medium, feed, or supplement powder or agglomerated powder; (b) combining the media powder or agglomerated powder with one or more lubricants, fillers, binders, or combinations thereof; and (c) producing cell culture medium, feed, or supplement tablets using a tableting apparatus. In one aspect, the cell culture medium, feed, or supplement is an agglomerated powder produced by fluid bed agglomeration. In another aspect, the cell culture medium, feed, or supplement powder or agglomerated powder is combined with a lubricant. In another aspect, the tableted composition comprises 95-99 percent by mass cell culture medium, feed, or supplement powder or agglomerated powder and 1-5 percent of one or more lubricants. In another aspect, the lubricant comprises magnesium stearate. In another aspect, the cell culture medium, feed, or supplement powder or agglomerated powder is combined with a lubricant and a disintegrant. In another aspect, the tableted composition comprises 95-99 percent by mass cell culture medium, feed, or supplement powder or agglomerated powder; 1-5 percent of one or more lubricants; and 1-5 percent of one or more disintegrants. In another aspect, the lubricant comprises magnesium stearate and the disintegrant comprises croscarmellose sodium. In another aspect, the tablet has a mass of 2.0 to 5.0 g. In another aspect, the tablet has a hardness of 18-22 kp. In another aspect, the tablet dissolves within 10-30 min in water at 25° C.

Another embodiment described herein is a tableted cell culture medium, feed, or supplement produced by any of the methods described herein. In one aspect, the tablet comprises: a media component comprising: 20-65 percent by mass carbohydrates; 20-40 percent by mass amino acids; 2-10 percent by mass inorganic salts or buffers; 1-5 percent by mass vitamins; 0.01-0.05 percent by mass trace minerals; and a tableting component comprising: 1-10 percent by mass of a lubricant, filler, binder, or combinations thereof. In another aspect, the tablet comprises 95-99 percent by mass cell culture medium, feed, or supplement; and 1-5 percent of one or more lubricants. In another aspect, the tablet comprises 90-99 percent by mass cell culture medium, feed, or supplement; 1-5 percent by mass of a disintegrant comprising croscarmellose sodium; and 1-5 percent by mass of a lubricant comprising magnesium stearate.

In another aspect, the tablet has a mass of 2.0 to 5.0 g. In another aspect, the tablet has a hardness of 18-22 kp. In another aspect, the tablet dissolves within 10-30 min in water at 25° C.

Another embodiment described herein is a kit comprising a package comprising one or more tablets of cell culture medium, feed, supplement, or buffer; a diluent; a receptacle suitable for reconstituting the one or more one or more tablets of cell culture medium, feed, supplement, or buffer; and instructions for use.

Another embodiment described herein is a method for making a cell culture medium, feed, supplement, or buffer from a tableted cell culture medium, feed, supplement, or buffer composition, the method comprising: (a) combining one or more tablets of cell culture medium, feed, supplement, or buffer with water until dissolved; (b) optionally, adding any supplements comprising amino acids, antibiotics, sera, or other cell culture media supplements; and (c) optionally, sterilizing the reconstituted cell culture medium, feeds, supplements, or buffers. In one aspect, the sterilization comprises filtration or gamma irradiation. In another aspect, the tablet dissolves within about 10-30 min in water at 25° C.

Another embodiment described herein is a cell culture medium, feed, supplement, or buffer prepared by a method described herein.

Another embodiment described herein is a system comprising a cell and a cell culture medium, feed, supplement, or buffer described herein.

Another embodiment described herein is a method of culturing a cell in a liquid reconstituted from a tableted cell culture medium comprising: reconstituting a tableted cell culture medium, feed, or supplement in a suitable liquid or buffer; and culturing the cell in the reconstituted medium under conditions favorable for growth.

Another embodiment described herein is a method of optimizing the concentration of a cell culture media component, the method comprising: measuring the concentration of one or more media components in a cell culture; determining whether the one or more media components are within an acceptable concentration range; if needed, supplementing the one or more media components in the cell culture with a tableted cell culture media composition. In one aspect, the measuring comprises a method selected from HPLC, mass spectrometry, ELISA, or standard curve assay. In another aspect, the supplementation comprises adding the tableted media composition direct to the culture or dissolving the tableted media composition in a solvent and then adding the dissolved tableted media composition to the cell culture.

Another embodiment described herein is the use of a tableted cell culture medium, feed, or supplement for preparing a cell culture medium, feed, or supplement.

Another embodiment described herein is the use of a tableted cell culture medium, feed, or supplement for culturing cells. In another aspect, the tableted cell culture medium, feed, or supplement for culturing is reconstituted and the cell cultured under favorable growth conditions.

### BRIEF DESCRIPTION OF THE DRAWINGS

FIG. 1A shows exemplary media tablets formed from CHO CD EFFICIENTFEED B AGT Nutrient Supplement (GIBCO) and varying amounts of magnesium stearate. The tablets contain about 2.5 g of media, are 16 mm×9.9 mm, and have a hardness of ˜19 kp. See Table 4.

FIG. 1B shows exemplary media tablets formed from CHO CD EFFICIENTFEED B AGT Nutrient Supplement (GIBCO) and 2 percent magnesium stearate and their disintegration in water at 25° C. The disintegration was conducted with 50 g (20 tablets) in 926 mL of water, which is the recommended media preparation procedure. Tablets containing 2 percent by mass magnesium stearate disintegrated within 28 min and tablets containing 5 percent by mass magnesium stearate disintegrated within 38 min. See Table 5.

FIG. 2A shows CHO DG44 LH cells grown in CD OPTICHO (GIBCO) in shake flasks and supplemented with glucose (negative control), CHO CD EFFICIENTFEED B AGT (GIBCO) (positive control), or CHO CD EFFICIENTFEED B AGT Tablets (“Tablet Feeds”) containing 2 percent or 5 percent by mass magnesium stearate. Cell cultures supplemented with Tablet Feeds showed viable cell counts (e.g., ˜14×10⁶ cells/mL) comparable to cell cultures supplemented with CHO CD EFFICIENTFEED B AGT (i.e., non-tableted feed; positive control). The unsupplemented cell cultures containing only glucose showed lower viable cell counts ˜10×10⁶ cells/mL (negative control) as compared to the cell cultures supplemented with CHO CD EFFICIENTFEED B AGT.

FIG. 2B shows the same data as FIG. 2A as a scatter plot.

FIG. 3A shows IgG titers from the cell cultures discussed in FIG. 2A-2B. Cell cultures supplemented with Tablet Feeds showed comparable IgG production (˜230 μg/mL) to cell cultures supplemented with CHO CD EFFICIENTFEED B AGT (i.e., non-tableted feed; positive control). The unsupplemented cell cultures containing only glucose showed lower IgG production (˜115 μg/mL) as compared to cell cultures supplemented with CHO CD EFFICIENTFEED B AGT.

FIG. 3B shows the same data as FIG. 3A in a scatter plot.

FIG. 4 shows a flow chart for the large-scale manufacturing process for the production of media tablets.

FIGS. 5A, 5B, 5C show physical parameters for 16.0 mm (2.4 g) CHO CD EFFICIENTFEED B AGT tablets. FIG. 5A shows tablet masses; FIG. 5B shows tablet thicknesses; and FIG. 5C shows tablet hardnesses, respectively, compared with control limits. See Tables 8-10.

FIGS. 6A, 6B, 6C show physical parameters for 22.0 mm (3.7 g) CHO CD EFFICIENTFEED B AGT Tablets. FIG. 6A shows tablet masses; FIG. 6B shows tablet thicknesses; and FIG. 6C shows tablet hardnesses, respectively, compared with control limits. See Tables 8-10.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry described herein are those that are known and commonly used in the art. In case of conflict, the present document, including definitions, will control. Preferred compositions, methods, or materials are described herein, although alternative compositions, methods, or materials similar or equivalent to those described herein can be used in practice or testing of the embodiments described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

All ranges disclosed include both end points as discrete values as well as all integers and fractions specified within the ranges with the same degree of precision is explicitly contemplated. For example, a range of 0.1-2.0 includes 0.1, 0.2, 0.3, 0.4 . . . 2.0. If the end points are modified by the term “about,” the range specified is expanded by a variation of up to ±10 percent of any value within the range, including the end points.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. The term “about” as used herein refers to any values, including both integers and fractional components that are within a variation of up to ±10 percent of the value modified by the term “about.” In certain aspects, the term “about” refers to a range of values that fall within 20 percent, 19 percent, 18 percent, 17 percent, 16 percent, 15 percent, 14 percent, 13 percent, 12 percent, 11 percent, 10 percent, 9 percent, 8 percent, 7 percent, 6 percent, 5 percent, 4 percent, 3 percent, 2 percent, 1 percent, or less in either direction (greater than or less than) of the stated reference value unless otherwise stated or otherwise evident from the context (except where such number would exceed 100 percent of a possible value). Alternatively, “about” can mean within 3 or more than 3 standard deviations, per the practice in the art. Alternatively, such as with respect to biological systems or processes, the term “about” can mean within an order of magnitude, in some embodiments within 5-fold, and in some embodiments within 2-fold, of a value. As used herein the symbol “˜” preceeding any value means “about.”

As used herein, the term “substantially” means to a great or significant extent, but not completely.

As used herein, the terms “control” or “reference” are used interchangeably and refer to a predetermined value or range, which is employed as a benchmark against which to assess the measured result.

The terms “agglomerated,” “Advanced Granulation Technology™,” or “AGT” (GIBCO) as used herein refer to granular dry media format produced through an advanced manufacturing process to produce complete media formulations in a variety of serum-free, protein-free, and chemically defined media in a dry format. The media is typically prepared using fluid bed technology that produces agglomerated powders having enhanced characteristics (for example, enhanced solubility) from the starting materials. The process consists of suspending powders in an upwardly moving column of air while simultaneously injecting a controlled and defined amount of liquid into the powder stream to produce a moistened state of the powder; mild heat may be then used to dry the material, producing an agglomerated powder. Preparation of agglomerated media, feeds, nutritive powders, supplements, etc., their properties, and methods to prepare auto pH and auto osmolality of agglomerated media, feeds, nutritive powders, supplements, etc. have been described in U.S. Pat. Nos. 6,383,810 and 6,627,426 and U.S. Pat. App. Pub. No. US 2019/0048312 A1, inter alia, each of which is incorporated by reference for teachings related to agglomerated media.

The terms “tablet” or “tableted” as used herein refer to a media, feed, media supplement, media subgroup, or buffer that has been compressed into a tablet format. Tablets differ from pellets in that they have a defined weight, may be rapidly mass-produced, and may contain conventional pharmaceutically acceptable excipients such as lubricants, binders, disintegrants, coatings, etc. See e.g., U.S. Pat. Pub. No. US 2018/0142203 A1. Additionally, the methods or processes for manufacturing tablets are different from pellets because tablets are made using compression processes whereas pellets are made using a rotary fluid bed process.

The term “powder” or “dry powder” as used herein refers to media powders or powdered media compositions for cell culture that are present in dry granular form, whose gross appearance may be free flowing. The term “powder” includes agglomerated powders, as described herein. The term “base powder” or “dry base powder” as used herein refers to a dry powder composition before it is tableted.

As used herein, the term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, carbohydrates, lipids, nucleic acids, hormones, vitamins, fatty acids, proteins and the like. Other ingredients that promote or maintain cultivation of cells ex vivo can be selected by those of skill in the art, in accordance with the particular need.

The terms “cell culture” or “culture” as used herein refer to the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms, for which the terms “tissue culture,” “organ culture,” “organ system culture” or “organotypic culture” may be used interchangeably with the term “cell culture.”

The phrases “cell culture medium,” “culture medium,” “medium formulation,” or “medium” (plural “media” in each case) as used herein refer to a nutritive solution that supports the cultivation and/or growth of cells; these phrases may be used interchangeably. A cell culture medium may be a basal medium (a general medium that requires additional ingredients to support cell growth) or a complete medium that has all or almost all components to support cell growth. Cell culture media may be serum-free, protein-free (one or both), may or may not require additional components like growth factors, additives, feeds, supplements, for efficient and robust cell performance.

Nutritive media can also be divided into various “subgroups” that can be prepared and used as described herein. Such subgroups can be combined to produce a nutritive medium. For examples of compatible subgroups and related considerations, see U.S. Pat. Nos. 5,474,931 and 5,681,748, which are incorporated by reference herein for such teachings.

The term “combining” as used herein refers to the mixing or admixing of ingredients in a cell culture medium formulation. Combining can occur in liquid or powder form or with one or more powders and one or more liquids. In another example, two or more powdered components may be mixed and then agglomerated to produce a complex mixture such as media, media supplements, media subgroups, or buffers. Combining also includes mixing dry components with liquid components.

The phrases “concentrate feed supplement medium” or “concentrated feed supplement medium” are used interchangeably and refer to a medium that comprises at least one component that is at a concentration higher than that desired in the cell culture medium to be supplemented.

The term “contacting” as used herein refers to the placing of cells to be cultivated into a culture vessel with the medium in which the cells are to be cultivated. The term “contacting” encompasses inter alia mixing cells with medium, perfusing cells with medium, pipetting medium onto cells in a culture vessel, and submerging cells in culture medium.

The term “cultivation” as used herein refers the maintenance of cells in an artificial environment under conditions favoring growth, differentiation, or continued viability, in an active or quiescent state, of the cells. Thus, “cultivation” may be used interchangeably with “cell culture” or any of its synonyms described above.

The term “culture vessel” as used herein refers to a receptacle for holding cells. The vessel may be glass, plastic, metal, or other material that can provide an aseptic environment for culturing, holding, or storing cells.

The term “extract” as used herein refers to a composition comprising or concentrated preparation of the subgroups of a substance, typically formed by treatment of the substance either mechanically (e.g., by pressure treatment) or chemically (e.g., by distillation, precipitation, enzymatic action or high salt treatment).

The term “effective amount” or “effective concentration” refers to an amount of an ingredient, which is available for use. One example is the amount of a vitamin in a culture medium, which is available to cells for use in biological processes normally associated with that vitamin. Thus, an effective amount includes the amount of a cell culture ingredient (e.g., a vitamin or sugar) available for a cell to metabolize. An effective amount of an ingredient can be determined, for example, from the knowledge available to one skilled in the art and/or by experimental determination.

A “feed” or “supplement” as used herein refers to a composition when added to cells in standard culture may be beneficial for its maintenance, or expansion, or growth, or viability, or affects its cell performance, or increases culture longevity or maintaining cells in a pseudo-stationary phase wherein product expression continues, or results in a significant increase in final product titer. A feed or supplement may be used interchangeably in this disclosure and refers to tableted and liquid formats (including agglomerated formats) of media components comprising one or more amino acids, sugars, vitamins, buffers, sometimes, peptides, hydrolysates, fractions, growth factors, hormones, etc. required to rebalance or replenish or to modulate the growth or performance of a cell in culture, or a cell culture system. A feed or supplement may be distinguished from a cell culture medium in that it is added to a cell culture medium that can culture a cell. As would be understood by one of skill in the art, sometimes a feed/supplement may comprise mainly those amino acids, sugars, vitamins, buffers, etc. required to rebalance or replenish or modulate the growth or performance of a cell in culture, or a cell culture system. A feed or supplement may or may not be concentrated or may be partially concentrated for certain components only.

A cell culture medium is composed of a number of ingredients and these ingredients vary from one culture medium to another. A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic acid. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See Banes et al., Methods for Preparation of Media, Supplements and Substrate for Serum-Free Animal Cell Culture, Alan R. Liss, N.Y. (1984), which is incorporated by reference herein in its entirety. The osmolality and/or pH, however, may differ in a 1× formulation compared to the culture medium, particularly when fewer ingredients are contained in the 1× formulation. The 1× concentration of any component is not necessarily constant across various media formulations. 1× might therefore indicate different concentrations of a single component when referring to different media. However, when used generally, 1× will indicate a typical working concentration commonly found in the types of media being referenced. A 1× amount is the amount of an ingredient that will result in a 1× concentration for the relevant volume of medium.

A “10× formulation” as used herein refers to a solution wherein each ingredient in that solution is about 10 times more concentrated than the same ingredient in the cell culture medium. For example, a 10× formulation of RPMI-1640 culture medium may contain, among other ingredients, 2.0 g/L L-arginine, 0.5 g/L L-asparagine, and 0.2 g/L L-aspartic acid (compare 1× formulation, above). A “10× formulation” may contain a number of additional ingredients at a concentration about 10 times that found in the 1× culture medium. As will be readily apparent, “20× formulation,” “25× formulation,” “50× formulation” and “100× formulation” designate solutions that contain ingredients at about 20-, 25-, 50- or 100-fold concentrations, respectively, as compared to a working 1× cell culture medium. Again, the osmolality and pH of the media formulation and concentrated solution may vary. See U.S. Pat. No. 5,474,931, which is directed to culture media concentrate technology and is incorporated by reference herein for such teachings.

As used herein “physiologic pH” is greater than about 4 and less than about 9. Other or particular pH values or ranges, e.g., minimum or maximum pHs of greater than 4.2, 4.5, 4.8, 5.0, 5.2, 5.5, 5.7, 5.8, 6.0, 6.2, 6.5, 6.7, 6.8, 7.0, 7.2, 7.4, 7.5, 7.8, 8.0, 8.2, 8.4, 8.5, 8.7, 8.8, etc. or from about 4.0 to about 9.0, from about 4.0 to about 5.0, from about 5.0 to about 6.0, from about 6.0 to about 7.0, from about 8.0 to about 9.0, from about 4.0 to about 6.0, from about 5.0 to about 7.0, from about 6.0 to about 8.0, from about 7.0 to about 9.0, from about 6.0 to about 9.0, or from about 4.0 to about 7.0 may also be used for dissolving supplements. Some supplements, though not preferred, may only be entirely soluble outside these ranges.

An “auto-pH” or “auto-pHing” medium, medium supplement, or buffer as described herein is a formulation which has been formulated such that, upon rehydration with a solvent, the resulting medium, medium supplement or buffer solution is at a desired pH and does not require adjustment of the pH with acid or base prior to use. For example, an auto-pH tableted culture medium that is formulated to be used at pH 7.4 will, upon rehydration with a solvent, be at pH 7.4 and therefore will be ready for immediate use without further adjustment of the pH.

The phrase “without significant loss of biological and biochemical activity” as used herein refers to a decrease of less than about 30 percent, preferably less than about 25 percent, more preferably less than about 20 percent, still more preferably less than about 15 percent, and most preferably less than about 10 percent, of the biological or biochemical activity of the nutritive media, media supplement, media subgroup, buffer, or sample of interest when compared to a freshly made nutritive media, media supplement, media subgroup, buffer, or sample of the same formulation.

As used herein a “solvent” is a liquid that dissolves or has dissolved another ingredient of the medium. Solvents may be used in preparing media, in preparing media tablets, in preparing tablets of subgroups, or supplements or other formulations, and in reconstituting a tablet or diluting a concentrate in preparation for culturing cells. Solvents may be polar, e.g., an aqueous solvent, or non-polar, e.g., an organic solvent. Solvents may be complex, i.e., requiring more than one ingredient to solubilize an ingredient. Complex solvents may be simple mixtures of two liquids such as alcohol and water or may be mixtures of salts or other solids in a liquid. Two, three, four, five, six, or more components may be necessary in some cases to form a soluble mixture. Simple solvents such as mixtures of ethanol or methanol and water are preferred because of their ease of preparation and handling.

As used herein, a “lubricant” is added to compositions that are pressed into tablets to aid in compaction of granules into tablets and ejection of a tablet from a die press. Examples of lubricants include magnesium stearate, calcium stearate, zinc stearate, sodium steady fumarate, stearic acid, talc, glyceryl behenate, or a combination thereof. In one aspect, the lubricant is magnesium stearate.

As used herein, a “glidant” is a tablet component that imparts a composition with enhanced flow properties. Examples of glidants include colloidal silica, fumed silica, or talc.

As used herein, a “disintegrant” is a tablet component that aids in hydration, disintegration, dispersion, and dissolution. Examples of disintegrants include crospovidone, croscarmellose sodium, alginic acid, microcrystalline cellulose, polacrilin potassium, sodium starch glycolate, starch, pregelatinized starch, or combinations thereof. In one aspect, the disintegrant is crospovidone (e.g., crosslinked homopolymers of N-vinyl-2-pyrrolidone) having a particular particle size. In another aspect, the disintegrant is sodium starch glycolate. In another aspect the disintegrant is croscarmellose sodium (e.g., AC-DI-SOL, DuPont).

As used herein, a “filler” or “diluent” is a component that adds bulkiness to a composition. Examples of fillers or diluents include lactose, lactose monohydrate, glucose, fructose, sucrose, sorbitol, mannitol, dicalcium phosphate dihydrate, cellulose, ethylcellulose, methylcellulose, microcrystalline cellulose, crospovidone, or a combination thereof. In one embodiment, the filler or diluent is microcrystalline cellulose. In another aspect, the filler or diluent is microcrystalline cellulose such as AVICEL PH-101 or PH-102 (FMC) that have particle sizes of 50 μm or 100 μm, respectively.

As used herein, a “binder” is a component that provides enhanced cohesion or tensile strength (e.g., hardness). Examples of binders include dibasic calcium phosphate, sucrose, corn (maize) starch, microcrystalline cellulose, and modified cellulose (e.g., hydroxymethyl cellulose).

As used herein a “coating agent” is a component that makes tablets smoother, controls the disintegration and release rate, and makes the tablet more resistant to the environment (extending its shelf life), or enhances the appearance. Examples of coating agents include sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein, or combinations thereof.

As used herein, a “colorant” is a component that imparts a composition with a desired color. Examples of colorants include commercially available pigments such as FD&C Blue # 1 Aluminum Lake, FD&C Blue #2, other FD&C Blue colors, titanium dioxide, iron oxide, and/or combinations thereof. Other examples of colorants include typical pH indicators that are used in tissue culture media (e.g., phenol red).

As used herein, a “surfactant” is a component that imparts compositions with enhanced solubility and/or wetability. Examples of surfactants include sodium lauryl sulfate (SLS), sodium stearyl fumarate (SSF), PLURONIC surfactants, ADOGEN 464, ALKANOL 6112, BRIJ surfactants, IGEPAL surfactants, poly(ethylene glycol) sorbitan tetraoleate, poly(ethylene glycol) sorbitol hexaoleate, sorbitan monopalmitate, NONIDET P-40, TRITON N-101, SPAN 80, TRITON X-100, TRITON X-114, TRITON X-405, TWEEN 20, TWEEN 40, TWEEN 60, TWEEN 85, ZONYL FS-300, ZONYL FSN, or a combination thereof.

The term “extended period of time” is meant a period of time longer than that for which the sample (e.g., pharmaceutical composition, nutritive medium, medium supplement, medium subgroup, or buffer) is stored. As used herein, an “extended period of time” therefore means about 1-36 months, about 2-30 months, about 3-24 months, about 6-24 months, about 9-18 months, or about 4-12 months, under a given storage condition, which may include storage at temperatures of about −70° C. to about 25° C., about −20° C. to about 25° C., about 0° C. to about 25° C., about 4° C. to about 25° C., about 10° C. to about 25° C., or about 20° C. to about 25° C. Assays for determining the biological or biochemical activity of pharmaceutical or clinical compositions, cell culture reagents, nutrients, nutritive media, media supplement, media subgroup, or buffers are well known in the art and are familiar to one of ordinary skill.

Some embodiments described herein are tablet compositions for nutritive media, cell culture media, feeds, media supplements, media subgroups, or buffer; methods for producing tablets of nutritive media, media supplements, feeds, media subgroups, or buffers; and the tableted products produced thereby. Tableted nutritive media, media supplements and media subgroups produced as described herein include any media, media supplement or media subgroup (serum-free or serum-containing) which may be used to support the growth of a cell, which may be a bacterial cell, a fungal cell (particularly a yeast cell), a plant cell or an animal cell (particularly an insect cell, a nematode cell or a mammalian cell, most preferably a human cell), any of which may be a somatic cell, a germ cell, a normal cell, a diseased cell, a transformed cell, a mutant cell, a stem cell, a precursor cell or an embryonic cell. Preferred such nutritive media include, but are not limited to, cell culture media, most preferably a bacterial cell culture medium, plant cell culture medium, or animal cell culture medium. Preferred media supplements include, but are not limited to, undefined supplements such as extracts of bacterial, animal or plant cells, glands, tissues or organs, particularly bovine pituitary extract, bovine brain extract and chick embryo extract; and biological fluids (particularly animal sera, and most preferably bovine serum, particularly fetal bovine, newborn calf or normal calf serum, horse serum, porcine serum, rat serum, murine serum, rabbit serum, monkey serum, ape serum or human serum, any of which may be fetal serum) and extracts thereof (more preferably serum albumin and most preferably bovine serum albumin or human serum albumin). Medium supplements may also include defined replacements such as STEMPRO LIPOMAX replacement, OPTIMAB KNOCK-OUT Serum Replacement (GIBCO, Thermo Fisher Scientific Inc.), and the like, which can be used as substitutes for the undefined media supplements described above. Such supplements may also comprise defined components, including but not limited to, hormones, cytokines, neurotransmitters, lipids, attachment factors, proteins, and the like.

In one embodiment, the tableted media, media supplements, media subgroups, or buffers, comprises agglomerated powders of media, media supplements, media subgroups, or buffers. In one aspect described herein, the agglomerated nutritive media, media supplements, media subgroups, and buffers are produced using fluid bed technology to agglomerate the solutions of media, media supplements, media subgroups, or buffers. Fluid bed technology is a process of producing agglomerated powders having altered characteristics (particularly, for example, solubility) from the starting materials. In general applications of the technology, powders are suspended in an upwardly moving column of air while at the same time a controlled and defined amount of liquid is injected into the powder stream to produce a moistened state of the powder; mild heat is then used to dry the material, producing an agglomerated powder. In one aspect, the agglomerated media, media supplements, media subgroups, or buffers is produced using the proprietary ADVANCED GRANULATION TECHNOLOY (AGT dry media format) (GIBCO). See Jayme et al., “A Novel Application of Granulation Technology to Improve Physical Properties and Biological Performance of Powdered Serum-Free Culture Media,” In: Shirahata et al. (eds) Animal Cell Technology: Basic & Applied Aspects, Vol. 12 (2002), Springer, Dordrecht. Specific commercially available agglomerated media, supplements, feeds, and additives include CD CHO AGT, CD OPTICHO AGT, CD FORTICHO AGT, VP-SFM AGT, OptiPro AGT, CD Hybridoma AGT, CHO CD EFFICIENTFEED A, B and C AGT, CHO CD EFFICIENTFEED A+, B+ and C+ AGT Nutrient Supplement, and FUNCTIONMAX TiterEnhancer Additive (all from GIBCO; the respective product descriptions are each incorporated by reference herein for such teachings).

The formulations and methods described herein can be used to prepare tablets of any nutritive media, media supplement, media subgroup, or buffer and stored for an extended period of time without significant loss of biological or biochemical activity.

Any nutritive medium, medium supplement, medium subgroup, or buffer may be formed into tablets or prepared by the methods described herein. Particularly preferred nutritive media, media supplements, and media subgroups that may be prepared as described herein include cell culture media, media supplements, and media subgroups that support the growth of animal cells, plant cells, bacterial cells, or yeast cells. Particularly preferred buffers that may be prepared as described herein include balanced salt solutions, which are isotonic for animal cells, plant cells, bacterial cells, or yeast cells.

Examples of animal cell culture media that may be prepared as described herein include, but are not limited to, DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, F10 Nutrient Mixture, F12 Nutrient Mixture, and cell-specific serum-free media (SFM) such as those designed to support the culture of keratinocytes, endothelial cells, hepatocytes, melanocytes, CHO cells, 293 cells, PerC6, hybridomas, hematopoetic cells, embryonic cells, neural cells etc. Specific chemically defined media products include CD CHO Medium (GIBCO), CD OPTICHO Medium (GIBCO), EX-CELL ADVANCED CHO Medium (Millipore Sigma-Aldrich), HYCLONE ACTIPRO (GE Healthcare Life Sciences). Specific feed supplements include CHO CD EFFICIENTFEED A (or B) AGT Nutritional Supplement (GIBCO), CD EFFICIENTFEED C AGT Nutrient Supplement (GIBCO), EFFICIENTFEED A+ AGT Supplement (GIBCO), EFFICIENTFEED B+ AGT Supplement (GIBCO), RESUREGE CD1 Supplement (GIBCO), HYCLONE Cell Boost Supplements (various versions) (GE Healthcare Life Sciences), EX-CELL ADVANCED CHO Feed 1 (Millipore Sigma-Aldrich), et al. Other media, media supplements, and media subgroups suitable for preparation are available commercially. Formulations for these media, media supplements and media subgroups, as well as many other commonly used animal cell culture media, media supplements and media subgroups are well-known in the art and are described in the literature and available from commercial suppliers, e.g., Thermo Fisher Scientific Inc., Life Technologies, Gibco, Invitrogen, et al.

Examples of plant cell culture media that may be prepared as described herein include, but are not limited to, Anderson's Plant Culture Media, CLC Basal Media, Gamborg's Media, Guillard's Marine Plant Culture Media, Provasoli's Marine Media, Kao and Michayluk's Media, Murashige and Skoog Media, McCown's Woody Plant Media, Knudson Orchid Media, Lindemann Orchid Media, or Vacin and Went Media. Formulations for these media, which are commercially available, as well as for many other commonly used plant cell culture media, are known in the art and available from commercial manufacturers.

Examples of bacterial cell culture media that may be prepared as described herein include, but are not limited to, Trypticase Soy Media, Brain Heart Infusion Media, Yeast Extract Media, Peptone-Yeast Extract Media, Beef Infusion Media, Thioglycollate Media, Indole-Nitrate Media, MR-VP Media, Simmons' Citrate Media, CTA Media, Bile Esculin Media, Bordet-Gengou Media, Charcoal Yeast Extract (CYE) Media, Mannitol-salt Media, MacConkey's Media, Eosin-methylene blue (EMB) media, Thayer-Martin Media, Salmonella-Shigella Media, and Urease Media. Formulations for these media, which are commercially available, as well as for many other commonly used bacterial cell culture media, are well-known in the art and may be found for example in the DIFCO & BBL Manual, 2nd ed. (Becton, Dickinson and Company, 2009) and in the Manual of Clinical Microbiology (American Society for Microbiology, Washington, D.C.).

Examples of fungal cell culture media, particularly yeast cell culture media, that may be prepared as described herein include, but are not limited to, Sabouraud Media and Yeast Morphology Media (YMA). Formulations for these media are commercially available and are known in the art.

One embodiment described herein is a tableted media, media supplement, media subgroup, or buffer. Exemplary tablet compositions are shown in Table 1. A minimum tablet composition comprises the media, media supplement, media subgroup, or buffer and a lubricant. Other tablet excipients such as disintegrants, fillers, glidants, coatings, colors, etc. can optionally be added to adjust the properties of the tablet, such as the dissolution or disintegration rate, and stability. In one aspect, the media, media supplement, media subgroup, or buffer is an agglomerated the media, media supplement, media subgroup. Surprisingly it was found that agglomerated media was capable of being compressed into tablets. The tablets dissolved in times comparable to typical agglomerated media. Small amounts of lubricant can be added (0.5-5 percent by mass) to improve the tableting process and prevent sticking to the tablet dies. Surprisingly, this amount of lubricant did not affect cell growth or protein production by mammalian cells cultured in the reconstituted tablet media. Media tablets provide a convenient method for storing and accurately dispensing media with precise weights. For example, 20 tablets of 2.5 g can be reconstituted with water to produce 1 L of media. This eliminates the need to weigh out dry powder or agglomerated media, reduces handling, saves time, and permits rapid scale-up.

Another embodiment is a tableted media composition comprising the media, media supplement, media subgroup, or buffer; a disintegrant, and a lubricant. In one aspect, the disintegrant is croscarmellose sodium. In another aspect, the lubricant is magnesium stearate.

In one aspect, the tableted media, media supplement, media subgroup, or buffer composition comprises about 60 percent to about 99 percent by mass of a media, media supplement, media subgroup, or buffer, and about 1 percent to about 40 percent by mass of one or more acceptable tableting excipients compatible with cell culture. In one aspect, the composition comprises one or more lubricants, one or more fillers or diluents, one or more disintegrants, or one or more other pharmaceutically acceptable excipients compatible with cell culture. In one aspect, the composition comprises a media, media supplement, media subgroup, or buffer and one or more lubricants. In one aspect, the tableted media, media supplement, media subgroup, or buffer comprises a composition as in Table 1.

TABLE 1
Exemplary Media/Supplement Tablet Formulations

| | | Mass |
|---|---|---|
| | | Percent |
| Component | Exemplary Species | (%) |
| Agglomerated media, dry | Various as described | 70-99 |
| powdered media, supplements, | herein | |
| buffers, clinical solutions, etc. | | |
| Lubricant | Magnesium stearate | 1-10 |
| Optional Disintegrant | Crospovidone, | 0-10 |
| | croscarmellose sodium | |
| Optional Filler | Microcrystalline cellulose, | 0-10 |
| | lactose monohydrate | |
| Optional excipients (glidants, | Various | 0-5 |
| colors, coatings, etc.) | | |

An exemplary media formulation is provided in Table 2. The exemplary media composition can be formulated as a liquid or dry powder that is reconstituted or dissolved in water prior to use. Liquid components, such as fetal bovine serum, are typically added after dissolution of the dry powder. The media can also be prepared as agglomerated granules using fluid bed technology. Dry powder media and agglomerated media can be compressed into tablets as described herein.

TABLE 2
Exemplary Media Formulations

| | | Mass |
|---|---|---|
| | | Percent |
| Component | Exemplary Species | (%) |
| Carbohydrate(s) | glucose, fructose, trehalose, etc. | 20-65 |
| (carbon source) | | |
| Amino Acids | alanine, arginine, aspartic acid, glutamic acid, | 20-40 |
| | glycine, histidine, isoleucine, methionine, | |
| | phenylalanine, proline, hydroxyproline, serine, | |
| | threonine, tryptophan, valine, tyrosine, cysteine, lysine | |
| Inorganic salts, buffers | salts of sodium, potassium, magnesium, | 2-10 |
| | calcium, ammonium, phosphate, carbonate, sulfate | |
| Vitamins | retinol (A), thiamine (B₁), riboflavin (B₂), | 1-5 |
| | niacinamide (B₃), pantothenic acid (B5), | |
| | pyridoxamine (B₆), biotin (B₇), folic acid (B₉) | |
| | cobalamin (B₁₂), ascorbic acid (C), | |
| | cholecalciferol (D), tocopherol (E), | |
| | phylloquinone (K), choline, inositol, lipoic acid, | |
| | para-aminobenzoic acid | |
| Trace minerals | iron, manganese, copper, iodine, zinc, cobalt, | 0.01-0.05 |
| | fluoride, chromium, molybdenum, selenium, | |
| | nickel, silicon, vanadium | |
| Serum or serum | fetal bovine serum (FBS), serum replacement | 1-10 |
| replacement | (e.g., KnockOut, GIBCO) | |
| Additives | antibiotics, pH indicators, surfactants, lipids, etc. | 0.1-5 |

The exemplary media formulation shown in Table 2 can be formulated into tablets by adding one or more of lubricants, fillers, or diluents, disintegrants, or other pharmaceutically acceptable excipients compatible with cell culture as shown in Table 1. In one aspect, the exemplary media is combined with at least 1-5 percent by mass of one or more lubricants to form tablets. In another aspect, the exemplary media is combined with at least 1-5 percent by mass of one or more lubricants and 1-5 percent by mass of one or more disintegrants to form tablets. In another aspect, the media is an agglomerated using fluid bed technology. The agglomerated media is then combined with at least 1-5 percent by mass of one or more lubricants and 1-5 percent by mass of one or more disintegrants and then tableted using standard tableting technology (e.g., a Korsh tableting press).

Any of the above media, media supplement, media subgroup, or buffer that can be prepared as described herein may also include one or more additional components, such as indicating or selection agents (e.g., dyes, antibiotics, amino acids, enzymes, substrates and the like), filters (e.g., charcoal), salts, polysaccharides, ions, detergents, stabilizers, and the like. The embodiment is not limited to presently formulated media but is broadly applicable to any media formulation or supplement for culturing cells.

In another embodiment described herein, the tableted culture media may comprise one or more buffer salts, preferably sodium bicarbonate, at concentrations sufficient to provide optimal buffering capacity for the culture medium. In one aspect the one or more buffer comprises acetic acid, acetylsalicylic acid, adipic acid, alginic acid, ascorbic acid, aspartic acid, benzoic acid, benzenesulfonic acid, bisulfic acid, boric acid, butanoic acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, gly-glycine, gluco heptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthalenesulfonic acid, naphthilic acid, nicotinic acid, nitrous acid, oxalic acid, pelargonic, phosphoric acid, propionic acid, pyruvic acid, saccharin, salicylic acid, sorbic acid, succinic acid, sulfuric acid, tartaric acid, thiocyanic acid, thioglycolic acid, thiosulfuric acid, tosylic acid, undecylenic acid, MES, bis-tris methane, ADA, ACES, bis-tris propane, PIPES, MOPSO, cholamine chloride, MOPS, BES, TES, HEPES, DI PSO, MOBS, acetamido glycine, TAPSO, TEA, POPSO, HEPPSO, EPS, HEPPS, Tricine, Tris(hydroxymethyl)aminomethane (tromethamine), glycinamide, glycylglycine, HEPBS, Bicine, TAPS, AMPB, CHES, AMP, AMPSO, CAPSO, CAPS, CABS, combinations thereof, or salts thereof. In one aspect, the buffer comprises one or more of phosphate, sulfate, carbonate, formate, acetate, propionate, butanoate, lactate, glycine, maleate, pyruvate, citrate, aconitate, isocitrate, α-ketoglutarate, succinate, fumarate, malate, oxaloacetate, aspartate, glutamate, tris(hydroxymethyl)aminomethane (tromethamine), combinations thereof, or salts thereof. In one aspect, the buffer is sodium carbonate or sodium bicarbonate.

According to one aspect described herein, a buffer salt, such as sodium bicarbonate, may be added to the medium prior to, during, or following agglomeration of the medium prior to tableting. In one example of this aspect described herein, the sodium bicarbonate may be added to the culture medium prior to, during or following agglomeration with an appropriate solvent (such as water, serum or a pH-adjusting agent such as an acid (e.g., HCl at a concentration of 1 M to 5 M, 0.1 M to 5 M, or preferably at 1 M) or a base (e.g., NaOH at a concentration of 1 M to 5 M, 0.1 M to 5 M, or preferably at 1 M) such that, upon reconstitution of the agglomerated medium the culture medium is at the optimal or substantially optimal pH for cultivation of a variety of cell types. For example, bacterial cell culture media prepared by the present methods will, upon reconstitution, preferably have a pH of about 4-10, more preferably about 5-9 or about 6-8.5. Fungal (e.g., yeast) cell culture media prepared by the present methods will, upon reconstitution, preferably have a pH of about 3-8, more preferably about 4-8 or about 4-7.5; animal cell culture media prepared by the present methods will, upon reconstitution, preferably have a pH of about 6-8 or about 7-8, more preferably about 7-7.5, or about 7.2-7.4; and plant cell culture media prepared by the present methods will, upon reconstitution, preferably have a pH of about 4-8, preferably about 4.5-7, 5-6 or 5.5-6. Of course, optimal pH for a given culture medium to be used on a particular cell type may also be determined empirically by one of ordinary skill using art-known methods. For example, gastric cells may be cultured at pHs well below those of other cells, for example, pH 1-3. One of ordinary skill appreciates that other cells adapted to harsh environments may have special tolerances or needs that might be outside the normal ranges that satisfy culture conditions for commonly cultured cells.

In another example, one or more buffer salts, e.g., sodium bicarbonate, may be added directly to a nutritive medium by tablet form. In a related aspect, a pH-adjusting agent such as an acid (e.g., HCl) or a base (e.g., NaOH) may be added to a nutritive medium, which may contain one or more buffer salts (such as sodium bicarbonate), by agglomeration of the pH-adjusting agent into nutritive medium in a fluid bed apparatus, by spray-drying the pH-adjusting agent onto the powdered or agglomerated nutritive medium, or by a combination thereof; this approach obviates the subsequent addition of a pH-adjusting agent after reconstitution of the powdered medium. This composition can then be formed into tablets as described herein. Thus, the tablet nutritive culture medium described herein is useful in cultivation or growth of cells in vitro that, upon reconstitution with a solvent (e.g., water or serum), has a pH that is optimal for the support of cell cultivation or growth without a need for adjustment of the pH of the liquid medium. This type of medium, defined herein as “automatically pH-adjusting medium,” therefore obviates the time-consuming and error-prone steps of adding buffer(s) to the medium after reconstitution and adjusting the pH of the medium after dissolution of the buffer(s). For example, a mammalian cell culture medium prepared according to these methods may, upon reconstitution, have a pH of between about 7.1 to about 7.5, more preferably between about 7.1 to about 7.4, and most preferably about 7.2 to about 7.4 or about 7.2 to about 7.3.

In another embodiment, automatically pH-adjusting media can be produced by preparing reconstituted media without the addition of any buffering systems or pH-adjusting agents. In a preferred such aspect, an auto-pH medium may be provided by adjusting the buffering systems present in the medium. For example, as one of ordinary skill is aware, culture media typically contain buffers or buffering systems. By adjusting the pH-opposing forms of such buffers in the medium, an auto-pH medium is created, avoiding the requirement to add additional buffers or pH-adjusting agents to achieve a proper pH level prior to or upon reconstitution of the medium and prior to use. In one such aspect described herein, pH-opposing forms of certain media components (particularly phosphate or other buffer salts) are then used in the culture medium to provide a desired pH upon reconstitution of the powdered media. pH-opposing forms of components are conjugate acid-base pairs in which the members of the pair can either raise the pH or lower it to achieve the desired pH of the solution. Sodium HEPES (pH raising) and HEPES-HCl (pH lowering) are examples of pH opposing components. For example, if a reconstituted media having a pH of between 4.5 and 7.2 is to be prepared, the first step is to determine the correct balance of monobasic (to lower the pH) to dibasic (to raise the pH) phosphate in order to yield the desired pH. Typically, mono- and di-basic phosphate salts are used at concentrations of about 0.1 mM to about 10 mM, about 0.2 mM to about 9 mM, about 0.3 mM to about 8.5 mM, about 0.4 mM to about 8 mM, about 0.5 mM to about 7.5 mM, about 0.6 mM to about 7 mM, or preferably about 0.7 mM to about 7 mM. If other buffer systems are used in the formulations, the proper ratio or balance of the basic (typically sodium or monobasic) buffer salt and the corresponding acidic (or pH-opposing; typically HCl or dibasic) buffer salt is similarly determined to ensure that the formulation will be at the desired final pH upon reconstitution with a solvent. Because the actual phosphate molecular species that is present in a solution is the same at a given pH whether the basic (e.g., sodium or monobasic) or acidic (e.g., HCl or dibasic) form is added, this adjustment would not be expected to impact buffering capacity. Once an appropriate ratio of pH-opposing forms of an appropriate buffer is determined, these components may be added to the medium (for example, a dry powder medium) to provide a culture medium that is of the appropriate pH level upon reconstitution and prior to use.

In one embodiment, the tableted media, media supplement, media subgroup, or buffer dissolves within about 10-30 minutes in water at 25° C. In one aspect, the time until complete dissolution of a tableted medium (e.g., full dissolution rate or t100) is within about 1 min, about 2.5 min, about 5 min, about 10 min, about 15 min, about 20 min, about 25 min, or about 30 min.

In another aspect, the full dissolution rate is within about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, or about 40 min.

In another embodiment, about 50 percent of the tableted media, media supplement, media subgroup, or buffer dissolves within about 10-30 minutes in water at 25° C. In one aspect, the time until 50 percent dissolution of a tableted medium (e.g., t50) is within about 1 min, about 2.5 min, about 5 min, about 10 min, about 15 min, about 20 min, about 25 min, or about 30 min. In another aspect, 50 percent dissolution rate is within about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, or about 40 min.

In another aspect, a single tableted medium completely dissolves within about 20-30 min in about 50 mL of water at 25° C. In another aspect, 20 media tablets completely dissolve within about 20-30 min in about 930 mL of water at 25° C. In one aspect, the disintegration is performed according to United States Pharmacopia (U.S.P.) Method (701) Disintegration, which is incorporated by reference herein for such teachings.

Another embodiment is tableted complete dry powder culture media formulations that support the cultivation of cells in vitro upon reconstitution of the media tablets with a solvent, without the need for the addition of any supplemental nutrient components to the medium prior to use. Media according to this aspect described herein thus will preferably comprise the nutritional components necessary for cultivation of a cell in vitro, such that no additional nutritional components need be included in the solvent or added to the medium upon reconstitution and prior to use. Accordingly, such complete media described herein will be suitable for use in cultivating cells in vitro upon reconstitution with water or with an alternative non-nutrient-containing solvent such as a buffered saline solution. Such complete media may be automatically pH-adjusting media, and may comprise one or more culture medium supplements (including but not limited to serum), one or more amino acids (including but not limited to l-glutamine), insulin, transferrin, one or more hormones, one or more lipids, one or more growth factors, one or more cytokines, one or more neurotransmitters, one or more extracts of animal tissues, organs or glands, one or more enzymes, one or more proteins, one or more trace elements, one or more extracellular matrix components, one or more antibiotics, one or more viral inhibitors, one or more buffers, or combinations thereof.

Examples of media supplements that may be prepared as tablets by the present methods, or that may be included in the culture media described herein, include, without limitation, animal sera, such as bovine sera, fetal bovine, newborn calf and calf sera, human sera, equine sera, porcine sera, monkey sera, ape sera, rat sera, murine sera, rabbit sera, ovine sera and the like, defined replacements such as STEMPRO LIPOMAX, OPTIMAB, KNOCK-OUT Serum Replacement (GIBCO, Thermo Fisher Scientific Inc.), hormones (including steroid hormones such as corticosteroids, estrogens, androgens (e.g., testosterone) and peptide hormones such as insulin, cytokines (including growth factors (e.g., EGF, αFGF, βFGF, HGF, IGF-1, IGF-2, NGF and the like), interleukins, colony-stimulating factors, interferons and the like), neurotransmitters, lipids (including phospholipids, sphingolipids, fatty acids, EXCYTE, cholesterol and the like), attachment factors (including extracellular matrix components such as fibronectin, vitronectin, laminins, collagens, proteoglycans, glycosaminoglycans and the like), and extracts or hydrolysates of animal, tissues (e.g., plant or bacteria tissues), cells, organs or glands (such as bovine pituitary extract, bovine brain extract, chick embryo extract, bovine embryo extract, chicken meat extract, chicken tissue extract, Achilles tendon and extracts thereof) and the like. Other media supplements that may be produced by the present methods or that may be included in the culture media described herein include a variety of proteins (such as serum albumins, particularly bovine or human serum albumins; immunoglobulins and fragments or complexes thereof; aprotinin; hemoglobin; haemin or haematin; enzymes (such as trypsin, collagenases, pancreatinin, or dispase); lipoproteins; fetuin; ferritin; etc.), which may be natural or recombinant; vitamins; amino acids and variants thereof (including, but not limited to, l-glutamine and l-cysteine), enzyme co-factors; polysaccharides; salts or ions (including trace elements such as salts or ions of molybdenum, vanadium, cobalt, manganese, selenium, and the like); and other supplements and compositions that are useful in cultivating cells in vitro that will be familiar to one of ordinary skill. Media supplements produced by the methods described herein include animal or mammalian (e.g., human, fish, bovine, porcine, equine, monkey, ape, rat, murine, rabbit, ovine, insect, etc.) derived supplements, ingredients, or products. These sera and other media supplements are available commercially. Alternatively, sera and other media supplements described herein may be isolated from their natural sources or produced recombinantly by art-known methods that will be routine to one of ordinary skill. See Freshney, R. I., Culture of Animal Cells, New York: Alan R. Liss, Inc., pp. 74-78 (1983), and references cited therein; see also Harlow, E., and Lane, D., Antibodies: A Laboratory Manual, Cold Spring Harbor Laboratory, N.Y, 116-120 (1988).

Components that are often present in the final formulation in μg/mL or even μg/L amounts have typically been left out of standard powdered media due to homogeneity and/or stability concerns, and instead are typically added to the reconstituted 1× media as a concentrate, thereby increasing storage costs and causing production of a finished culture medium to become more costly and less efficient. In one aspect, such low-level components may be added to the base powder by first making a concentrate of the components and then spraying them into a portion of the powdered media that would be granulated with the concentrate. This would then be milled to a particle size in the same general size range as that of the bulk for blending. The ability to spray-in components in small amounts may be especially helpful in developing media that include trace elements, vitamins, viral inhibitors, growth factors, cytokines, and the like. Specifically, among others, the components to be added to a powdered medium include but are not limited to vitamins comprising retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, para-aminobenzoic acid, salts thereof, and trace elements including iron, manganese, copper, iodine, zinc, cobalt, fluoride, chromium, molybdenum, selenium, nickel, silicon, vanadium, salts thereof, or combinations thereof. Additional components to be added in low amounts to the culture media described herein may include, for example, growth factors (e.g., EGF, αFGF, βFGF, KGF, HGF, IGF-1, IGF-2, NGF, insulin, and the like), interleukins, colony-stimulating factors, interferons, attachment factors, extracellular matrix components (e.g., collagens, laminins, proteoglycans, glycosaminoglycans, fibronectin, vitronectin, and the like), lipids (such as phospholipids, cholesterol, bovine cholesterol concentrate, fatty acids, sphingolipids and the like); extracts of animal tissues, glands or organs; antibiotics such as GENETICIN carbenicillin, cefotaxime, anti-PPLO, FUNGIZONE, hygromycin, kanamycin, neomycin, nystatin, penicillin, or streptomycin, etc.; and viral inhibitors (e.g., protease inhibitors, nucleoside analogues, and the like, which are known in the art).

Examples of buffers that may be prepared as described herein and/or that may be included in the culture media include, but are not limited to, buffered saline solutions, phosphate-buffered saline (PBS) formulations, Tris-buffered saline (TBS) formulations, HEPES-buffered saline (HBS) formulations, Hanks' Balanced Salt Solutions (HBSS), Dulbecco's PBS (DPBS), Earle's Balanced Salt Solutions, Puck's Saline Solutions, Murashige and Skoog Plant Basal Salt Solutions, Keller's Marine Plant Basal Salt Solutions, Provasoli's Marine Plant Basal Salt Solutions, and Kao and Michayluk's Basal Salt Solutions, and the like. Formulations for these buffers, which are commercially available, as well as for many other commonly used buffers, are well-known in the art and may be found for example in the Thermo Fisher Scientific Catalog, in the DIFCO & BBL Manual, 2n ed. (Becton, Dickinson and Company, 2009), and in the Millipore Sigma Cell Culture Catalogues.

Tablets of clinical solutions, particularly those used for parenteral nutrition, electrolyte balance or intravenous (IV) solutions are also described. Such clinical solutions include but are not limited to Ringer's, Ringer's lactate, 5 percent by mass dextrose in water, normal saline (0.9 percent by mass NaCl), hypotonic saline (0.45 percent by mass NaCl), 5 percent by mass dextrose in saline, and the like. Clinical solutions may further comprise one or more pharmaceutical compositions or components thereof.

Also described is a method for preparing tableted nutritive media, media supplements, media subgroups, buffers, or samples that contain a desired or effective amount or concentration of an ingredient(s), wherein at least one ingredient such as a sugar (e.g., glucose) vitamins, an amino acid, a salt, a trace element, a growth factor and/or an amine (e.g., ethanolamine, spermine, spermidine, putrescene or para-aminobenzoic acid) is inputted into the manufacturing process at a higher amount as compared to the final product. The method of compensating for a loss or decrease in effective concentration of at least one ingredient during an agglomeration process comprising calculating or determining the amount of the ingredient to be added to the process as described herein to result in the final desired or effective amount.

One method for determining the effective concentration of a compound (e.g., a vitamin) in a test culture medium is as follows. Using a vitamin for the purposes of illustration, a known concentration of the vitamin is serially diluted into a culture medium lacking the vitamin. A second set of serial dilutions are set-up where the test culture medium is serially diluted into a culture medium also lacking the vitamin. Cells that require the vitamin for growth are then added to both sets of serially diluted samples and cultured under appropriate conditions. After a period of time, cell replication is measured (e.g., by cell counting or by measuring optical density). The measurements of the known concentrations are graphed to form a standard curve, to which the measurements from the test culture medium dilutions are compared to determine the effective concentration of the vitamin in the test culture medium. Any number of similar assays may be used to determine the amount of a metabolite(s) in a sample, which are available for cellular metabolism.

Another embodiment is a method for sterilizing the nutritive media, media supplements, media subgroups and buffers described herein, as well as for sterilizing powdered nutritive media, media supplements, media subgroups and buffers prepared by standard methods such as ball-milling or lyophilization. Also described are methods for sterilizing or substantially sterilizing the samples including nutritive media, media supplements, media subgroups, and buffers described herein. Such additional methods may include filtration, heat sterilization, irradiation, or other chemical or physical methods. Tableted nutritive media, media supplements, media subgroups, or buffers (prepared as described herein may be irradiated under conditions favoring sterilization. Since nutritive media, media supplements, media subgroups, and buffers are usually prepared in large volume solutions and frequently contain heat labile components, they are not amenable to sterilization by irradiation or by heating. Thus, nutritive media, media supplements, media subgroups, and buffers are commonly sterilized by contaminant-removal methods such as filtration which significantly increases the expense and time required to manufacture such media, media supplements, media subgroups, and buffers.

Tableted nutritive media, media supplements, media subgroups, or buffers prepared according to the methods described herein can be sterilized by less expensive and more efficient methods. For example, powdered nutritive media, media supplements, media subgroups, or buffers may be irradiated under conditions favoring sterilization of these powders. Preferably, this irradiation is accomplished in bulk (i.e., following packaging of the sample, nutritive media, media supplement, media subgroup, or buffer), and most preferably this irradiation is accomplished by exposure of the bulk packaged sample, media, media supplement, media subgroup, or buffer described herein to a source of gamma rays under conditions such that bacteria, fungi, spores or viruses that may be resident in the powdered sample media, media supplements, media subgroups, or buffers are inactivated (i.e., prevented from replicating). Alternatively, irradiation may be accomplished by exposure of the sample, powdered media, media supplement, media subgroup, or buffer, prior to packaging, to a source of gamma rays or a source of ultraviolet light. The sample, media, media supplements, media subgroups and buffers described herein may alternatively be sterilized by heat treatment (if the subgroups, or components of the sample, nutritive media, media supplement, media subgroup, or buffer are heat stable), for example by flash pasteurization or autoclaving. As will be understood by one of ordinary skill in the art, the dose of irradiation or heat, and the time of exposure, required for sterilization will depend upon the bulk of the materials to be sterilized, and can easily be determined by the ordinarily skilled artisan without undue experimentation using art-known techniques, such as those described herein.

In one embodiment described herein, the bulk sample (e.g., nutritive media, media supplements, media subgroups, or buffers) (which are preferably in powdered form) are exposed to a source of irradiation (e.g., γ (gamma) radiation) at a total dosage of about 10-100 kilograys (kGy), preferably a total dosage of about 15-75 kGy, 15-50 kGy, 15-40 kGy, 20-40 kGy or 25-45 kGy, more preferably a total dosage of about 20-30 kGy, and most preferably a total dosage of about 25-35 kGy, for about 1 hour to about 7 days, more preferably about 1 hour to about 5 days, 1 hour to about 3 days, about 1-24 hours or about 1-5 hours, and most preferably about 1-3 hours (“normal dose rate”). Alternatively, the bulk powders described herein may be sterilized at a “slow dose rate” of a total cumulative dosage of about 25-100 kGy over a period of about 1-5 days. During irradiation, the nutritive media, media supplements, media subgroups, or buffers (which are preferably in powdered form) are preferably stored at a temperature of about −70° C. to about room temperature (about 20-25° C.), most preferably at about −70° C. One of ordinary skill will appreciate, of course, that radiation dose and exposure times may be adjusted depending upon the bulk and/or mass of material to be irradiated; typical optimal irradiation dosages, exposure times and storage temperatures required for sterilization of powdered materials by irradiation or heat treatment are known in the art.

Following sterilization, unpackaged tableted nutritive media, media supplements, media subgroups and buffers may be packaged under aseptic conditions, for example by packaging into containers such as sterile tubes, vials, bottles, bags, pouches, boxes, cartons, drums and the like, in vacuum packaging, or integrated powder/solvent packaging described herein. Sterile packaged samples such as media, media supplements, media subgroups, and buffers may then be stored for extended periods of time as described herein.

The tableted nutritive media, media supplements, media subgroups, and buffers described herein are readily soluble in a rehydrating solvent and are substantially dust free. For use, the tableted media, media supplement, media subgroup, or buffer may be “rehydrated” or “reconstituted” in a volume of a solvent sufficient to produce the desired nutrient, electrolyte, ionic and pH conditions required for the particular use of the solvated media, media supplement, media subgroup, or buffer. This reconstitution is particularly facilitated because the tableted media, media supplements, media subgroups, and buffers will readily dissolve and will produce little if any dust or insoluble material, unlike powdered nutritive media, media supplements, media subgroups, or buffers.

Solvents for use in reconstituting the tableted nutritive media, media supplements, media subgroups, buffers, or samples described herein include, but are not limited to, the solvents described herein such as water, such as distilled and/or deionized water, serum (bovine or human serum and most particularly fetal bovine serum or calf serum), organic solvents (dimethylsulfoxide, acetone, ethanol and the like), or any combination thereof, any of which may contain one or more additional components (e.g., salts, polysaccharides, ions, detergents, stabilizers, etc.). For example, tableted media supplements (such as animal sera) and buffers are preferably reconstituted in water to a 1× final concentration, or optionally to a higher concentration (e.g., 2×, 2.5×, 5×, 10×, 20×, 25×, 50×, 100×, 500×, 1000×, etc.) for the preparation of stock solutions or for storage. Alternatively, tableted culture media may be reconstituted in a solution of media supplements (e.g., sera such as FBS) in water, such as those solutions wherein the media supplement is present at a concentration, for example, of 0.5 percent, 1 percent, 2 percent, 2.5 percent, 5 percent, 7.5 percent, 10 percent, 15 percent, 20 percent, 25 percent, 50 percent, or higher, by volume (vol/vol) in the water.

Reconstitution of the tableted sample (e.g., nutritive media, media supplements, media subgroups, or buffers) is typically accomplished under aseptic conditions to maintain the sterility of the reconstituted sample, although the reconstituted sample may be sterilized by filtration or other sterilization methods that are well known in the art, following rehydration. Following their reconstitution, media, media supplements, media subgroups and buffers or other samples should be stored at temperatures below about 10° C., preferably at temperatures of about 0-4° C., until use.

The reconstituted tableted nutritive media, media supplements, media subgroups and buffers may be used to culture or manipulate cells according to standard cell culture techniques that are known to one of ordinary skill in the art. In such techniques, the cells to be cultured are contacted with the reconstituted media, media supplement, media subgroup, or buffer described herein under conditions favoring the cultivation or manipulation of the cells (such as controlled temperature, humidity, lighting, and atmospheric conditions). Cells that are particularly amenable to cultivation by such methods include, but are not limited to, bacterial cells, fish cells, yeast cells, plant cells, and animal cells. Such bacterial cells, yeast cells, plant cells and animal cells are available commercially from known culture depositories, e.g., the American Type Culture Collection (Manassas, Va.) and others that will be familiar to one of ordinary skill in the art. Preferred animal cells for cultivation by these methods include, but are not limited to, insect cells (most preferably Drosophila cells, Spodoptera cells and Trichoplusia cells), nematode cells (most preferably C. elegans cells) and mammalian cells (most preferably CHO cells, COS cells, VERO cells, BHK cells, AE-1 cells, SP2/0 cells, L5.1 cells, hybridoma cells and human cells, such as 293 cells, PER-C6 cells and HeLa cells), any of which may be a somatic cell, a germ cell, a normal cell, a diseased cell, a transformed cell, a mutant cell, a stem cell, a precursor cell or an embryonic cell, embryonic stem cells (ES cells), cells used for virus or vector production (i.e., 293, PerC 6), cells derived from primary human sites used for cell or gene therapy, i.e., lymphocytes, hematopoietic cells, other white blood cells (WBC), macrophage, neutrophils, dendritic cells, and any of which may be an anchorage-dependent or anchorage-independent (i.e., “suspension”) cell. Another aspect is the manipulation or cultivation of cells and/or tissues for tissue or organ transplantation or engineering, i.e., hepatocyte, pancreatic islets, osteoblasts, osteoclasts/chondrocytes, dermal or muscle or other connective tissue, epithelial cells, tissues like keratinocytes, cells of neural origin, cornea, skin, organs, and cells used as vaccines, i.e., blood cells, hematopoietic cells other stem cells or progenitor cells, and inactivated or modified tumor cells of various histotypes.

Another embodiment is a method of manipulating or culturing one or more cells comprising contacting said cells with the cell culture reagents described herein, particularly reconstituted tableted nutritive media, media supplement, media subgroup, or buffer and incubating said cell or cells under conditions favoring the cultivation or manipulation of the cell or cells. Any cell may be cultured or manipulated according to the present methods, particularly bacterial cells, yeast cells, plant cells, animal cells and other cells or cell lines described herein. Cells cultured or manipulated according to this aspect described herein may be normal cells, diseased cells, transformed cells, mutant cells, somatic cells, germ cells, stem cells, precursor cells or embryonic cells, any of which may be established cell lines or obtained from natural sources.

Tableted nutritive media, media supplements and media subgroups produced by the present methods are any media, media supplement or media subgroup (serum-free or serum-containing) which may be used to manipulate or support the growth of a cell, which may be a bacterial cell, a fungal cell (particularly a yeast cell), a plant cell, or an animal cell (particularly an insect cell, a nematode cell, or a mammalian cell, most preferably a human cell), any of which may be a somatic cell, a germ cell, a normal cell, a diseased cell, a transformed cell, a mutant cell, a stem cell, a precursor cell, or an embryonic cell. Preferred such nutritive media include, but are not limited to, cell culture media, most preferably a bacterial cell culture medium, plant cell culture medium or animal cell culture medium. Preferred media supplements include, but are not limited to, undefined supplements such as extracts or hydrolysates of bacterial, animal or plant cells, glands, tissues or organs (particularly bovine pituitary extract, bovine brain extract and chick embryo extract); and biological fluids or blood derived products (particularly animal sera, and most preferably bovine serum (particularly fetal bovine, newborn calf, or normal calf serum), horse serum, porcine serum, rat serum, murine serum, rabbit serum, monkey serum, ape serum, or human serum, any of which may be fetal serum) and extracts thereof (more preferably serum albumin and most preferably bovine serum albumin or human serum albumin). Medium supplements may also include defined replacements such as STEMPRO LIPOMAX replacement, OPTIMAB replacement, KNOCK-OUT Serum Replacement (GIBCO, Thermo Fisher Scientific Inc.), and the like, which can be used as substitutes for the undefined media supplements described above. Such supplements may also comprise defined components, including but not limited to, hormones, cytokines, neurotransmitters, lipids, attachment factors, proteins, amino acids, and the like.

The tableted media described herein, upon being reconstituted with a solvent, can be used for the growth and/or cultivation of organisms such as, e.g., filamentous fungi, transgenic plants (e.g., tobacco, rice, and Lemna), lichens, or algae, or cells derived from any of the aforementioned organisms.

Also described are tableted forms of media supplements or feeds. In one aspect, the supplement includes one or more amino acids. In another aspect, a salt of an amino acid is used. In another aspect, the salt is a sodium salt. In another aspect, monobasic and dibasic phosphate salts are used. A preferred cation is sodium. In another aspect, the monobasic and dibasic salts are provided such that a resultant pH, for example, about 8 pH is obtained. Depending on the formulation, while the ratio of monobasic to dibasic salts may be dictated by desired pH, different total salt concentrations should be tried to optimize solubility, especially when concentrated or highly concentrated supplements are to be used. The pH can also be confirmed when assessing the salt concentration. When an amino acid is not provided as a salt, preferably the pH effect of the acid is countered by a tribasic phosphate, preferably a sodium tribasic phosphate. While sodium is preferred as a cation, other metals, such as potassium, calcium, magnesium may be used. If a specific counter ion is desired, it may be available as a phosphate salt. In another aspect, the supplement powder dissolves rapidly. In another aspect, the supplement can be prepared and used as a highly concentrated mixture, for example, with one or more components at a concentration about 2× or more, preferably 3×, 5×, 8×, 10×, 12×, 15×, 20×, 25×, 50×, 75×, 85×, 95×, or even about 10033 or more times the concentration of that component in the medium being supplemented. The concentration of each desired ingredient of the supplement can be independently selected. In another aspect, the supplement is prepared by reconstituting with water under sterile conditions. In another aspect, the supplement is sterilized by filtration.

A supplement may have no ingredients in common with the medium being supplemented or may have one or more ingredients in common. The supplement may differ from the medium being supplemented in at least one manner, such as a different concentration of one or more ingredients, for example a different ratio of two ingredients, a different ingredient mix, additional ingredients, or omitted ingredients in the supplement. For example, a supplement may omit salts to the extent feasible and may contain, for example, significantly enhanced concentrations of growth factors or amino acids. A preferred supplement formulation contains at least 2, more preferably 3, but perhaps at least 4, 5, 6, 7, 8, 9, 10, 11, 12, 13, 14, 15, or more amino acids including salts, or dimers thereof.

In some embodiments, feed supplements as described herein are utilized to supplement a medium that has or is being used to culture cells, e.g., as the cells are cultured, some ingredients are removed from the medium by the cells. In some embodiments described herein, the feed supplement is used, inter alia, to replace some or all of these ingredients. In some embodiments, the supplement contains the majority of the ingredients that were in the original medium to be supplemented, but the feed medium is lacking at least one ingredient. In some embodiments, the feed supplement is lacking 1, 2, 3, 4, 5, 6, 7, 8, 9, 10, 11, 12, 13, 14, 15, or more ingredients as compared to the concentration in the original culture medium being supplemented. In some embodiments, the feed supplement is added in a concentrated form, e.g., at 2×, 3×, 4×, 5×, 6×, 7×, 8×, 9×, 10×, 15×, 20×, 30×, 40×, 50×, 100×, 200×, 300×, 400×, 500× or 1000×. By concentrated form is meant that at least one of the ingredients in the feed supplement is at a concentration higher than what is the desired concentration in the culture medium. In some embodiments, ingredients for a feed supplement may be divided into multiple feed supplement media, e.g., based upon compatible subgroups.

In one embodiment, the feed supplement is in tablet form. The tableted feed supplement is typically reconstituted prior to the feeding. However, the addition of a sterile tableted supplement directly to the liquid medium is possible. Typically, in this case, the feed is set-up so that the tablet undergoes sufficient dissolution or mixing before contacting the cells.

In another embodiment, the tableted media, supplement, feed, subgroup, or buffer is added to a culture to optimize the concentration of a particular component, supplement a depleted or omitted component, or replace a component that has been consumed or degraded by the culture. The tablet can be dissolved in a solvent prior to addition to the culture or added in solid form.

In another embodiment, a cell culture is analyzed to determine the concentration of one or more media components. Typical analysis methods can be used to determine the concentration of a component such as HPLC, mass spectrometry, ELISA, standard curve assays, and other methods known in the art. If the concentration of a component is less than a desired range, a tableted supplement may be added to the culture to increase the concentration to the desired range. The tablet can be dissolved in a solvent prior to addition to the culture or added in tablet form and dissolved in situ.

In some embodiments, a portion of the ingredients of a feed supplement is reconstituted from a tablet, e.g., an agglomerated tableted supplement. In some embodiments, additional ingredients are added to the reconstituted media or a liquid form of a tableted supplement or feed. In some embodiments, the tableted additional ingredients comprise amino acids or antibiotics.

Osmolality (a measure of osmotic pressure) of cell culture medium is important as it helps regulate the flow of substances in and out of the cell. It is typically controlled by the addition or subtraction of salt in a culture medium. Rapid increases in osmolality (e.g., addition of concentrated feed supplement with elevated osmolality relative to the base growth medium) may result in stressed, damaged, or dead cells. Maintaining an optimal osmolality range during cell culture/growth is desirable for cell function and/or bioproduction success.

Base growth medium osmolality generally ranges from 250 mOsmo/kg to 350 mOsmo/kg. In some embodiments, addition of a concentrated feed supplement described herein increases osmolality by about 25 mOsmo/kg or by between from about 0 to about 100, about 0.01 to about 100, about 0.1 to about 100, about 1 to about 100, about 10 to about 100, about 50 to about 100, about 75 to about 100, about 1 to about 10, about 1 to about 50, about 1 to about 75, about 10 to about 50, about 15 to about 35, about 25 to about 50, or about 20 to about 30 mOsmo/kg. In some embodiments, the osmolality of a concentrated feed supplement medium described herein (e.g., a 5× concentrated feed supplement medium) has an osmolality between from about 0 to about 1500; 1 to about 1000; 1 to about 750; 1 to about 500; 1 to about 400; 1 to about 300; 1 to about 200; 1 to about 100; 1 to about 50; 50 to about 1000; 100 to about 1000; 300 to about 1000; 500 to about 1000; 750 to about 1000; 100 to about 200; 200 to about 300; 300 to about 400; 400 to about 500; 450 to about 500; 500 to about 600; 550 to about 650; 600 to about 700; 750 to about 850; 700 to about 800; 800 to about 900; 900 to about 1000; 1000 to about 1250; or about 1250 to about 1500 mOsmo/kg. In some embodiments, the osmolality of a concentrated feed supplement medium described herein is between from about 3.0× to about 3.5×, about 3.5× to about 4.5×, about 4.5× to about 5.5×, about 5.5× to about 6.5×, about 6.5× to about 7.5×, about 7.5× to about 8.5×, about 8.5× to about 9.5×, about 9.5× to about 10.5×, about 10.5× to about 11.5×, about 11.5× to about 12.5×, about 12.5× to about 13.5×, about 13.5× to about 14.5×, about 14.5× to 18.5 to about 19.5×, about 19.5× to about 20.5×, about 3× to about 10×, about 5× to about 10×, about 10× to about 15×, about 15× to about 20×, about 20× to about 25×, or about 25× to about 100× as compared to the osmolality of the medium being supplemented or fed.

Described herein are methods for the preparation of tableted nutritive media, media supplements, media subgroups, buffers, and cells at reduced cost. The compositions and methods provide for the preparation of tableted nutritive media, media supplements, media subgroups, buffers, and cells at reduced cost and reduced inconvenience. The cost reductions are due to the several factors. For example, the tableted media, media supplement, media subgroup, and buffer formulations may be produced with much smaller production facilities since the large stir tanks required for 1× formulations are not required. In addition, the tableted media, media supplement, media subgroup and buffer formulations may be prepared on an as needed basis using “just in time” production techniques, which reduce inventory, storage, and labor costs. The time required for the preparation and shipping of the media, media supplement, media subgroup and buffer formulations may be reduced from 6-8 weeks to as little as one day. The automatically pH-adjusting tableted media described herein also provide significant cost and time savings and reduce the tendency for introduction of contamination into reconstituted media that may occur during the pH adjustment process according to standard methods using traditional dry powder or bulk liquid media.

Also described is the preparation of tableted nutritive media, media supplements, media subgroups, or buffers which may be used to prepare very large quantities of 1× media, media supplements, media subgroups, or buffers (e.g., 100,000 liters or more) which would require only one quality control test compared to multiple quality control tests for multiple batches produced according to other commonly used techniques. Importantly, the tableted media, media supplement, media subgroup, or buffer formulations are more consistent between batches since the individual components are more stable. Further, the tableted media, media supplement, media subgroup, or buffer formulations can easily be dispensed to produce a particular volume without having to weigh the dry component. Each tablet is reconstituted to a particular volume. Thus, it is trivial to scale up and make non-standard volumes of the media, media supplement, media subgroup, or buffer as needed. Further, the tablets minimize dust and handling during reconstitution which prevents exposure and potential contamination.

Another embodiment described herein is a method for producing a tableted form of an agglomerated nutritive medium powder, an agglomerated medium supplement powder, an agglomerated nutritive medium subgroup powder, or an agglomerated buffer powder, said method comprising agglomerating a nutritive medium powder, medium supplement powder, nutritive medium subgroup powder, or buffer powder, with a solvent comprising at least one lipid dissolved therein, said solvent delivering said at least one lipid for incorporation in said nutritive medium powder, medium supplement powder, nutritive medium subgroup powder, or buffer powder. In one aspect, the agglomerating comprises fluid bed agglomeration. The agglomerated nutritive medium powder, medium supplement powder, nutritive medium subgroup powder, or buffer powder is then combined with one or more of a lubricant, filler, binder, or combinations thereof and compressed into a tablet.

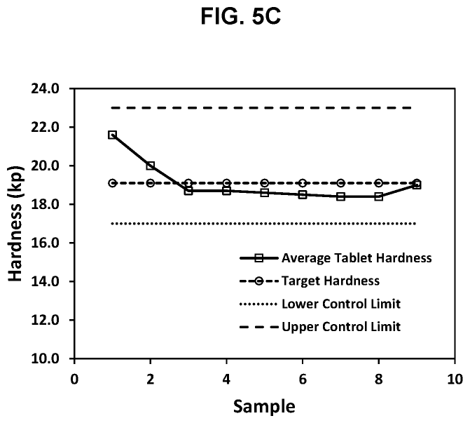

One embodiment described herein is a method for producing a tableted cell culture medium, feed, or supplement composition comprising: (a) preparing a cell culture medium, feed, or supplement powder or agglomerated powder; (b) combining the media powder or agglomerated powder with one or more lubricants, fillers, binders, or combinations thereof; and (c) producing cell culture medium, feed, or supplement tablets using a tableting apparatus. An exemplary manufacturing process is shown in FIG. 4. An exemplary manufacturing process is shown in FIG. 4. In one embodiment, the shape, size and weight of the tablet can be controlled due to the, for example, individualized production via the tableting apparatus. A tableting apparatus includes, for example, a KORSH PH100 Rotary Tablet Press, and others (see Examples).

The tableted media, media supplements, media subgroups, buffers, cells, and cell-containing compositions described herein are ideally suited for preparation of kits. Such a kit may comprise one or more containers such as vials, test tubes, bottles, packages, pouches, drums, and the like. Each of the containers may contain one or more of the tableted cell culture reagents, nutritive media, media supplements, media subgroups, cells, or buffers described herein, or combinations thereof. Such tableted cell culture reagents, nutritive media, media supplements, media subgroups, buffers or cells may be hydrated or dehydrated but are typically dehydrated preparations produced by the methods described herein. Such preparations may be sterile or substantially sterile.

A first container may contain, for example, a tableted nutritive media, media supplement, media subgroup, or a buffer described herein, or any component or subgroup thereof, such as any of those nutritive media, media supplements, media subgroups, or buffers described herein that are described herein. Additional tableted, dry, or liquid nutritive media, buffers, extracts, supplements, components, or subgroups may be contained in additional containers in the present kits. The kits may also contain, in one or more additional containers, one or more cells such as bacterial cells, yeast cells, plant cells, or animal cells. Such cells may be lyophilized, dried, frozen, or otherwise preserved, or may be spray-dried according to the methods described herein or treated by the method described herein. In addition, the kits described herein may further comprise one or more additional containers, containing, for example, l-glutamine, optionally complexed with one or more divalent cations. The kits may further comprise one or more additional containers containing a solvent to be used in reconstituting the dry powder pharmaceutical, or clinical compositions, cell culture reagents, nutritive media, media supplements, media subgroups and/or buffers; such solvents may be aqueous or organic and include buffer solutions, saline solutions, nutritive medium solutions, nutritive medium supplement solutions including sera such as bovine sera, fetal bovine sera, calf sera, human sera, or combinations thereof. Other ingredients that are not compatible for admixture with the nutritive media, buffers, pharmaceutical compositions, extracts, supplements, components, or subgroups described herein may be contained in one or more additional containers to avoid mixing of incompatible components. An exemplary kit may comprise a container containing a media tablet for reconstitution optionally of a volume sufficient to contain the reconstituting solvent, instructions for reconstitution and means for accessing the tablet(s) such as a tear strip or a port for introducing the reconstituting solvent.

The number and types of containers contained in a given kit (e.g., for making a nutritive medium, medium supplement, medium subgroup, or buffer) may vary depending on the desired product or the type of pharmaceutical or clinical compositions, media, media supplement, media subgroup, or buffer to be prepared. Typically, the kit will contain the respective containers containing the components or supplements necessary to make a particular pharmaceutical or clinical composition, media, media supplement, media subgroup, or buffer. However, additional containers may be included in the kit described herein so that different pharmaceutical or clinical compositions, media, media supplements, media subgroups, or buffers can be prepared by mixing different amounts of various components, supplements, subgroups, buffers, solvents, etc., to make different pharmaceutical or clinical compositions, media, media supplement, media subgroup, or buffer formulations.

It will be apparent to one of ordinary skill in the relevant art that suitable modifications and adaptations to the compositions, formulations, methods, processes, and applications described herein can be made without departing from the scope of any embodiments or aspects thereof. The compositions, methods, and experiments provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

### EXAMPLES

### Example 1

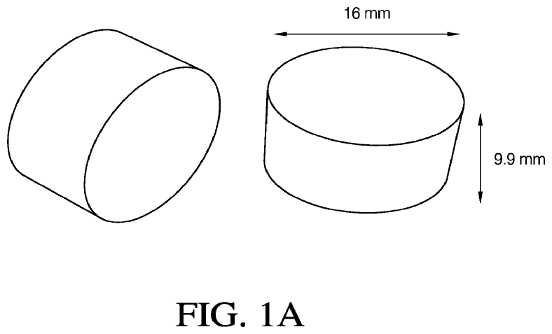

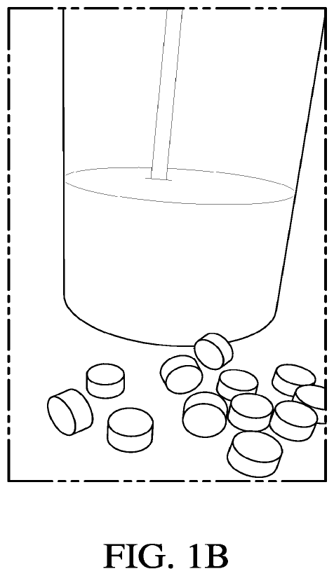

A typical CHO media, CHO CD EFFICIENTFEED B AGT Nutrient Supplement (GIBCO), was evaluated for the ability to be compressed into tablets. The media was analyzed for its material properties. See Table 3. Tablets were produced by loading 500 g of media into a manual 2-station Carver tablet press. The tablets had masses of 2.5-2.6 g and had 16 mm (⅝ in) diameter by 9.9 mm. See FIG. 1A. Tablet disintegration was performed using a standard powder disintegration apparatus that uses a slow up/down agitation. See FIG. 1B. The tablets were added to water at a ratio of 50 g (20 tablets) per 926 mL of water. This ratio of media to water is the recommended dissolution rate for the standard CHO CD EFFICIENTFEED B AGT Nutrient Supplement. The 20 tablets disintegrated within about 22-23 min at 25° C. (n=3 experiments).

TABLE 3
AGT Media Analysis and Tablet Properties
CHO CD EFFICIENTFEED B AGT Nutrient Supplement (GIBCO)

| | |
|---|---|
| Description | Pink granules |
| Solubility/Clarity | Soluble/Clear in aqueous solution |
| Bulk Density | 0.65 g/mL |
| Tap Density | 0.75 g/mL |
| Compressibility Index | 13 (good flowability) |
| Particle Size Distributions | |
| Mean Particle Size | 260 μm |
| Particle Size >150 μm | 57 percent |
| Particle Size <150 μm | 43 percent |
| Weight per tablet (16.0 mm, | 2.5-2.6 g (⅝″ diameter) |
| type B) Carver Press | |
| Disintegration Time: 20 tablets | 22-23 min |
| (water, 25° C.; n = 3) | |

### Example 2

The effect of using a tablet lubricant, magnesium stearate, was evaluated. Four specific trials were performed with CHO CD EFFICIENTFEED B AGT Nutrient Supplement (GIBCO) media and varying amounts of, magnesium stearate from 1 percent to 5 percent by mass.

TABLE 4
Exemplary AGT Media Tablet Formulations
CHO CD EFFICIENTFEED B AGT Nutrient Supplement (GIBCO)

| Formulation | Media (g) | Magnesium Stearate (g) |
|---|---|---|
| A - 1 percent magnesium | 495 | 5 |
| stearate | | |
| B - 2 percent magnesium | 490 | 10 |
| stearate | | |
| C - 3 percent magnesium | 485 | 15 |
| stearate | | |
| D - 5 percent magnesium | 475 | 20 |
| stearate | | |

For each formulation, various amounts of granulated media were blended with screened magnesium stearate as a lubricant to achieve the desired mass percentage in 500 g total mass. See Table 4. The blended media lubricant mixture was compressed into tablets (n=3) on a Korsch PH 100 tablet press (16.0 mm die), in order to achieve maximum tablet weight, optimum hardness, and thickness.

The blended media lubricant mixture containing 1 percent by mass magnesium stearate stuck to the lower punch and tablet die walls upon compression. The media blends with ≥2 percent by mass magnesium stearate did not stick to the tablet press.

The media tablets were analyzed for disintegration using a conventional propeller mixer and water at room temperature ˜25° C. at a ratio of 50 g (20 tablets) per 926 mL of water. See Table 5. This ratio of media to water is the recommended dissolution rate for the standard CHO CD EFFICIENTFEED B AGT Nutrient Supplement.

TABLE 5
Tablet Compression and Dissolution Results

| | Weight | Hardness | Thickness | Disintegration | |
|---|---|---|---|---|---|
| Formulation | (mg) | (kp) | (mm) | (min) | Observations |
| A | 2438 | 20.0 | 9.9 | Not performed | Sticking |
| (1 percent magnesium | 2530 | 20.1 | 9.9 | | |
| stearate by mass) | 2511 | 19.0 | 9.9 | | |
| B | 2345 | 19.5 | 9.9 | 28 min | No sticking |
| (2 percent magnesium | 2535 | 17.5 | 9.9 | | |
| stearate by mass) | 2498 | 19.2 | 9.9 | | |
| C | 2416 | 19.3 | 9.9 | Not performed | No sticking |
| (3 percent magnesium | 2456 | 19.3 | 9.9 | | |
| stearate by mass) | 2498 | 20.1 | 9.9 | | |
| D | 2500 | 19.2 | 9.9 | 38 min | No sticking |
| (5 percent magnesium | 2437 | 18.4 | 9.9 | | |
| stearate by mass) | 2397 | 19.8 | 9.9 | | |

### Example 3

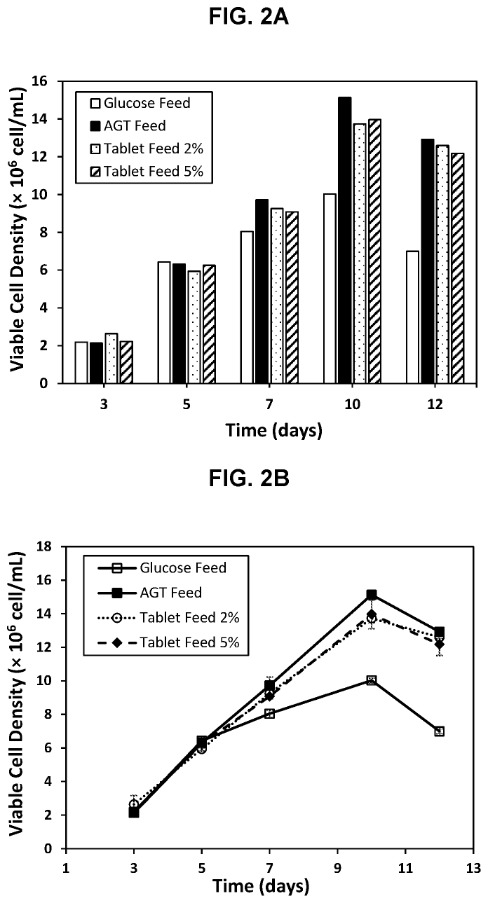

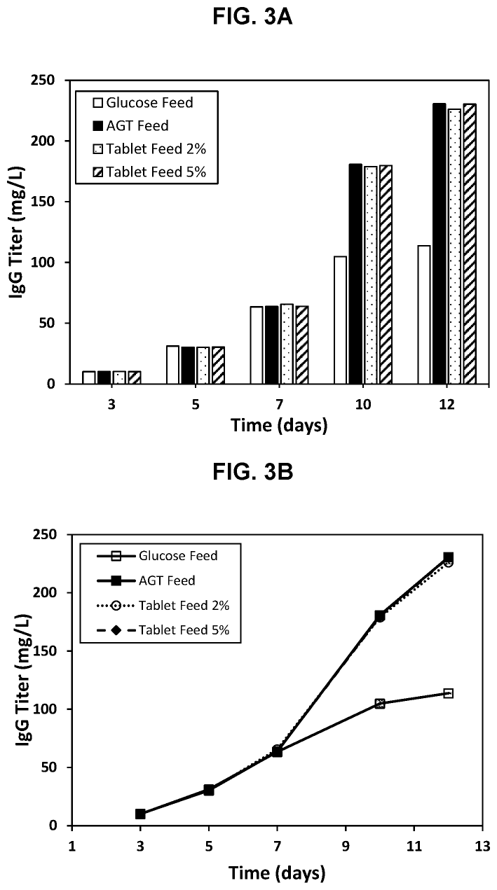

The media tablets were evaluated for cell growth and immunoglobulin productivity using batch fed 60 mL shake flasks. CHO DG44 LH cells were cultured in CD OPTICHO (GIBCO) for a minimum of 3 passages. The cultures were supplemented with glucose (negative control), CHO CD EFFICIENTFEED B AGT (GIBCO) (positive control), or CHO CD EFFICIENTFEED B AGT Tablets (“Tablet Feeds”) containing 2 percent or 5 percent by mass magnesium stearate. The tablets were reconstituted in water at a rate of 50 g (20 tablets) per 926 mL of water, filtered through a 0.1 μm filter, and used without further manipulation. The tablet reconstitution rate is comparable to the preparation of CD EFFICIENTFEED B AGT. Samples were taken every 2 days for a 12-day period to monitor cell viability and IgG production. Cell cultures supplemented with Tablet Feeds showed viable cell counts (e.g., ˜14×10⁶ cells /mL) comparable to cell cultures supplemented with CHO CD EFFICIENTFEED B AGT (i.e., non-tableted feed; positive control). The unsupplemented cell cultures containing only glucose showed lower viable cell counts ˜10×10⁶ cells /mL (negative control) as compared to the cell cultures supplemented with CHO CD EFFICIENTFEED B AGT. See FIG. 2A-2B.

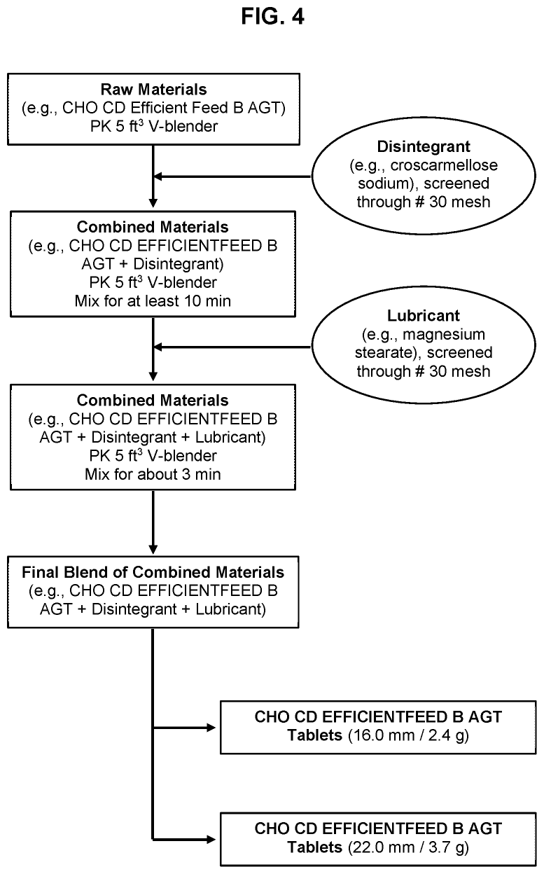

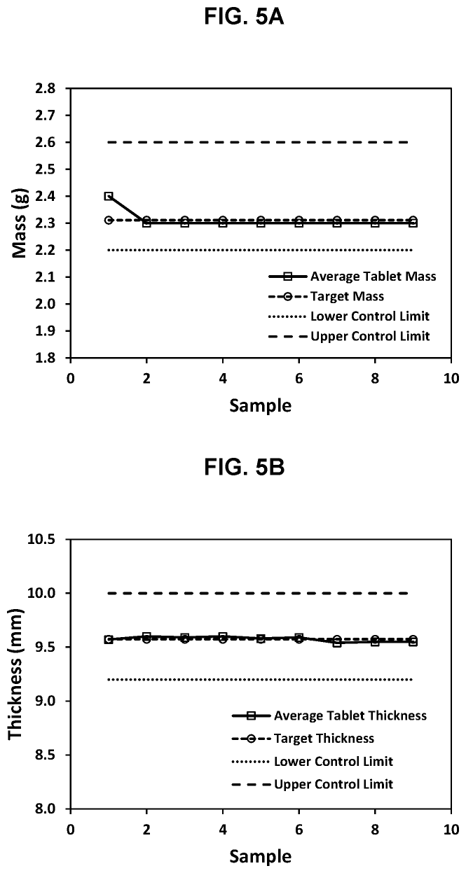

The cell cultures supplemented with Tablet Feeds showed comparable IgG production (˜230 μg/mL) to cell cultures supplemented with CHO CD EFFICIENTFEED B AGT (i.e., non-tableted feed; positive control). The unsupplemented cell cultures containing only glucose showed lower IgG production (˜115 μg/mL) as compared to cell cultures supplemented with CHO CD EFFICIENTFEED B AGT. See FIG. 3A-3B.

### Example 4

A DOE study was performed to identify a formula that would produce quality tablets for B and D type tooling using a Korsch PH 100 tablet Press. Each trial was conducted by manual blending of active granules CHO CD EFFICIENT B AGT and excipients. The blend was then compressed into tablet using Carver Press (Single Station) manually. Tablets were then evaluated for mass, thickness, hardness, friability, and disintegration time. The disintegration test was carried out using ˜900 mL of water (temp 25-30° C.) in beaker and propeller mixer. Friability was carried out using 1 tablet in a Friability Drum for 100 drops (4 min at 25 rpm).

Table 6 summarizes the physical data generated from the Carver Tablet Press (Manual Compression machine, Single Station) design of experiment (DOE) study conducted with CHO CD EFFICIENTFEED B AGT Tablets.

TABLE 6
CHO CD EFFICIENTFEED B AGT Tablet Formulations
Average Tablet Mass: 2.35-2.40 g; Average Thickness 9.9-10.1 mm

| | | | Compr. | | Disint. | | |
|---|---|---|---|---|---|---|---|
| | | Mass | Pres. | Hardn. | Time | Friab. | |
| No. | Materials | Percent | (psi) | (kp) | (min). | (%) | Comments |
| 1 | CHO CD EFFICIENTFEED | 92 | 1500 | 11 | N/P | NP | Slow disintegration, |
| | Granules | | | | | | |
| | Sodium starch glycolate | 5 | 2000 | 10 | NP | NP | high friability at |
| | Magnesium stearate | 3 | 2500 | 14 | 20 | | 18 kp |
| | | | 3000 | 18 | 25 | ~10 | |
| | Total | 100 | | | | | |
| 2 | CHO CD EFFICIENTFEED | 87 | 2700 | 12 | 22 | ~10 | Low hardness, |
| | Granules | | | | | | slow disintegration, |
| | Sodium starch glycolate | 10 | | | | | and high friability |
| | Magnesium stearate | 3 | | | | | at 12 kp |
| | Total | 100 | | | | | |
| 3 | CHO CD EFFICIENTFEED | 82 | 2700 | ~6 | ~22 | | Disintegration |
| | Granules | | psi | | | | acceptable but |
| | Croscarmellose sodium (AC-DI-SOL) | 15 | 3500 | ~9 | 14 | ~8 | low hardness |
| | Magnesium stearate | 3 | 4000 | ~10 | 13 | ~7 | and high friability |
| | | | 4500 | ~10 | 14 | ~7 | |
| | Total | 100 | | | | | |
| 4 | CHO CD EFFICIENTFEED | 87 | 4000 | ~12 | 14 | ~8 | Disintegration |
| | Granules | | | | | | acceptable but |
| | Croscarmellose sodium (AC-DI-SOL | 10 | | | | | low hardness |
| | Magnesium stearate | 3 | | | | | and high friability |
| | Total | 100 | | | | | |
| 5 | CHO CD EFFICIENTFEED | 87 | 4000 | ~17 | ~15 | 2 | Disintegration |
| | Granules | | | ~18* | | | and hardness |
| | Microcrystalline cellulose | 5 | | | | | acceptable; |
| | (AVICEL PH-101) | | | | | | friability slightly |
| | Croscarmellose sodium (AC-DI-SOL | 5 | | | | | high (edge chipped) |
| | Magnesium stearate | 3 | | | | | |
| | Total | 100 | | | | | |
| 6 | CHO CD EFFICIENTFEED | 92 | 4000 | ~22 | ~15 | 1-2 | Disintegration |
| | Granules | | | ~14* | | | and hardness |
| | Microcrystalline cellulose | 2.5 | | | | | acceptable; |
| | (AVICEL PH-101) | | | | | | friability slightly |
| | Croscarmellose sodium (AC-DI-SOL | 2.5 | | | | | high (edge chipped) |
| | Magnesium stearate | 3 | | | | | |
| | Total | 100 | | | | | |
| 7 | CHO CD EFFICIENTFEED | 92 | 4000 | ~17 | ~15 | 1-2 | Disintegration |
| | Granules | | | ~17* | | | and hardness |
| | Croscarmellose sodium (AC-DI-SOL | 5 | | | | | acceptable; |
| | Magnesium stearate | 3 | | | | | friability slightly |
| | Total | 100 | | | | | high (edge chipped) |
| 8 | CHO CD EFFICIENTFEED | 94.5 | 4000 | ~21 | 16 | 0 | Disintegration, |
| | Granules | | | ~24* | | | hardness, and |
| | Croscarmellose sodium (AC-DI-SOL | 2.5 | | | | | friability |
| | Magnesium stearate | 3 | | | | | acceptable |
| | Total | 100 | | | | | |

*Hardness after 24 hours

Table 7 summarizes the physical data generated from small scale trials on KORSH PH100 Rotary Tablet Press based on the DOE study conducted with CHO CD EFFICIENTFEED B AGT Tablets.

These small-scale compression trials using both “B” Type (16.0 mm) and “D” Type (22.0 mm) dies on a KORSH PH100 Rotary Tablet Press identified the critical quality attributes of the tablets before proceeding to larger batch sizes on KORSH PH 100 Rotary Tablet Press.

TABLE 7
CHO CD EFFICIENTFEED B AGT Tablet Physical Data

| | Punch | Avg. | Avg. | Avg. | | Disinteg. | |
|---|---|---|---|---|---|---|---|
| | Diam. | Mass | Thickness | Hardn. | Friability | Time | |
| Tooling | (mm) | (g) | (mm) | (kp) | (%) <1%* | (min.) | Comments |
| B | 16 | 2.4 | 9.6 | 19-21 | 0.36; 0.38 | 17; 19 | No issues |
| D | 22 | 3.7 | 8.8 | 18-20 | 0.75; 0.69 | 18; 20 | No issues |

*USP Guidance (1216) Tablet Friability

### Example 5

Scale-up Tablet Compression of Dry Format Cell Culture Medium Feed

Table 8 summarizes the composition of large-scale batches of 16.0 mm and 22.0 mm tablets made using a high speed KORSH PH100 Rotary Tablet Press.

TABLE 8
CHO CD EFFICIENTFEED B AGT 16.0 mm (2.2
g) and 22.00 mm (3.7 g) Tablets

| | Mass per | Mass | |
|---|---|---|---|
| Component | Tablet | Percent | Function |
| CHO CD EFFICIENTFEED B AGT | 2.268 | 94.5 | API |
| Granules | | | |
| Croscarmellose sodium (AC-DI-SOL) | 0.06 | 2.5 | Disintegrant |
| Magnesium stearate NF | 0.072 | 3.0 | Lubricant |
| Total | 2.4 | 100 | — |
| CHO CD EFFICIENTFEED B AGT | 3.5 | 94.5 | API |
| Granules | | | |
| Croscarmellose sodium (AC-DI-SOL) | 0.09 | 2.5 | Disintegrant |
| Magnesium stearate NF | 0.11 | 3.0 | Lubricant |
| Total | 3.7 | 100 | — |

Two different batch sizes 18.0 kg and 35.95 kg were selected for blending and compression of 2.4 g and 3.7 g tablets, respectively. The following batches were completed as part of the scale up. All batches were manufactured based on predetermined specifications that were established during small scale lab trials on CHO CD EFFICIENTFEED B AGT; 16.0 mm (2.4 g) and 22.0 mm (3.7 g). A general process description is shown in FIG. 4 and described below.

Screening

A disintegrating agent, croscarmellose sodium (AC-DI-SOL) was screened using a #30-mesh stainless steel screen. Next, a lubricant, magnesium stearate was screened using a #30-mesh stainless steel screen.

Blending

Approximately half of CD CHO EFFICIENTFEED B AGT raw materials were charged into a fixed speed PK 5 ft3 V-blender (24 rpm) followed by the screened croscarmellose sodium (AC-DI-SOL). The remaining quantity of CHO CD EFFICIENTFEED B AGT raw materials were added into the blender and blended for ten (10) minutes. The screened magnesium stearate was then added to the blender and mixed for three (3) minutes to yield the final blend.

Compression

The final blend was then compressed in a six (6) station Korsh PH100 Rotary Tablet Press.

Table 9 summarizes the quantities of starting powder and tablets produced during large scale batch production of 16.0 mm and 22.0 mm tablets made using a high speed KORSH PH100 Rotary Tablet Press.

TABLE 9
Scale-up Batch Sizes for 16.0 mm and 22.0 mm Tablets

| Tablet Size | | | Blend | Compression |
|---|---|---|---|---|
| (Mass) | Batch Size | | Batch | Batch |
| 16.0 mm (2.2 g) | 18 kg | 7,5000 tablets | 019 | 021 |
| 22.00 mm (3.7 g) | 35.98 kg | 9,724 tablets | 020 | 022 |

Table 10 summarizes physical parameters of the 16 mm (2.4 g) tablets and 22.0 mm (3.7 g) tablets.

TABLE 10
Manufacturing and Physical Parameters of the 16
mm (2.4 g) Tablets and 22.0 mm (3.7 g) Tablets
16.0 mm (2.4 g) Tablets

| | |
|---|---|
| Punch Size and Shape: | “16.0 mm” Round, Standard Plain Concave |
| | (“B” Type Tooling) |
| Average Tablet Weight | 2.40 g (2.30-2.50 g) |
| (10 tablets): | |
| Individual Weight Range: | 2.20-2.60 g |
| Tablet Hardness Target: | 20.0 kp |
| Tablet Hardness Range: | 17.0-23.0 kp |
| Tablet Thickness Range: | 9.6 mm (9.2-10.0 mm) |
| Tablet Press: | Korsh PH100, Force-feed, 6 Stations |
| Tablet Press Speed Range: | 15-20 RPM |

22.0 mm (3.7 g) Tablets

| | |
|---|---|
| Punch Size and Shape: | “22.0 mm ” Round, Standard Plain Concave |
| | (“D” Type Tooling) |
| Average Tablet Weight | 3.70 g (3.60-3.80 g) |
| (10 tablets): | |
| Individual Weight Range: | 3.50-3.90 g |
| Tablet Hardness Target: | 20.0 kp |
| Tablet Hardness Range: | 17.0-23.0 kp |
| Tablet Thickness Range: | 8.8 mm (8.5-9.1 mm) |
| Tablet Press: | Korsh PH100, Force-feed, 6 Stations |
| Tablet Press Speed Range: | 15-20 RPM |

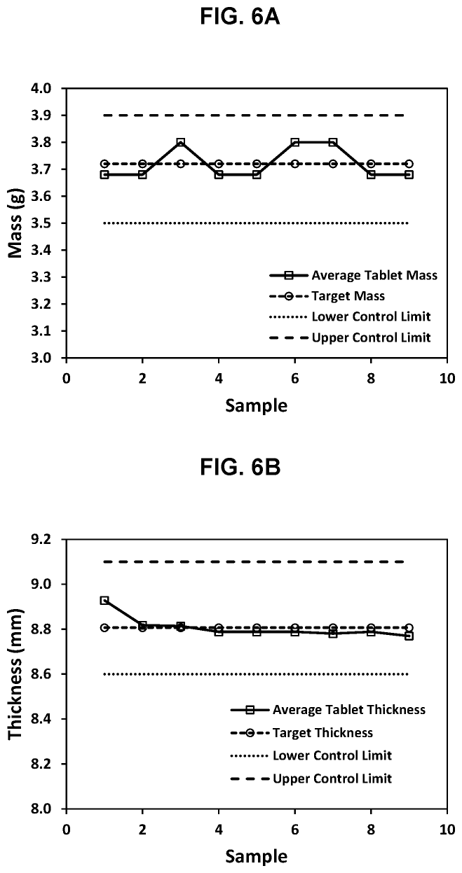

FIGS. 5A, 5B, and 5C show the tablet weight, thickness, and hardness, respectively, for the 16.0 mm (2.4 g) tablets shown in Table 9, Batch 22.

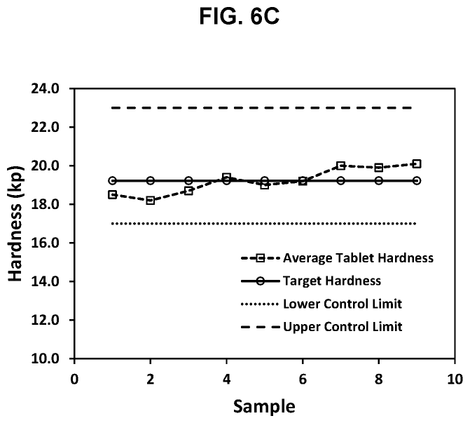

FIGS. 6A, 6B, and 6C show the tablet weight, thickness, and hardness, respectively, for the 22.0 mm (3.7 g) tablets shown in Table 9, Batch 21.

1. A tableted composition comprising a cell culture medium, feed, or supplement comprising:
amino acids, salts, buffers, trace minerals, vitamins, carbohydrates, lipids, nucleic acids, proteins; and
a lubricant, filler, binder, or combination thereof.

2. The composition of claim 1, wherein the tableted composition dissolves within about 10-30 minutes in water at 25° C.

3. The composition of claim 1, wherein the composition comprises:
95-99 percent by mass of amino acids, salts, buffers, trace minerals, vitamins, carbohydrates, lipids, nucleic acids, proteins; and
1-5 percent by mass of a lubricant comprising magnesium stearate.

4. The composition of claim 1, wherein the composition comprises:
90-99 percent by mass of amino acids, salts, buffers, trace minerals, vitamins, carbohydrates, lipids, nucleic acids, proteins; and
1-5 percent by mass of a disintegrant comprising croscarmellose sodium; and
1-5 percent by mass of a lubricant comprising magnesium stearate.

5. A composition comprising a tableted cell culture medium, feed, or supplement composition comprising:
a media component comprising:
20-65 percent by mass carbohydrates;
20-40 percent by mass amino acids;
2-10 percent by mass inorganic salts or buffers;
1-5 percent by mass vitamins;
0.01-0.05 percent by mass trace minerals; and
a tableting component comprising:
1-10 percent by mass of a lubricant, filler, binder, or combinations thereof.

6. The composition of claim 5, wherein the composition comprises:
(a) 20-65 percent by mass of mono or disaccharides comprising glucose, fructose, lactose, trehalose, maltose, or sucrose;
(b) 20-40 percent by mass of amino acids comprising alanine, arginine, aspartic acid, glutamic acid, glycine, histidine, isoleucine, methionine, phenylalanine, proline, hydroxyproline, serine, threonine, tryptophan, valine, tyrosine, cysteine, lysine, salts thereof, or combinations thereof;
(c) 2-10 percent by mass of salts of sodium, potassium, magnesium, calcium, ammonium, phosphate, carbonate, sulfate, or combinations thereof;
(d) 1-5 percent by mass of vitamins comprising retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, para-aminobenzoic acid, salts thereof, or combinations thereof;
(e) 0.01-0.05 percent by mass of trace elements including iron, manganese, copper, iodine, zinc, cobalt, fluoride, chromium, molybdenum, selenium, nickel, silicon, vanadium, salts thereof, or combinations thereof;
(f) 1-5 percent by mass of magnesium stearate; and
(g) 1-5 percent by mass of croscarmellose sodium.

7. The composition of claim 5, wherein the media component comprises an agglomerated dry powder.

8. The composition of any one of claims 5-7, wherein the tableted medium dissolves within about 10-30 minutes in water at 25° C.

9. The composition of any one of claims 5-8, wherein the tableted medium has a hardness of about 18-22 kp.

10. The composition of any one of claims 5-9, wherein the tableted medium has a mass of about 2.0 to 5.0 g.

11. The composition of any one of claims 5-10, wherein the tableted medium upon reconstitution with water exhibits cell viability levels and expressed protein levels commensurate with an analogous non-tableted medium.

12. A method for producing a tableted cell culture medium, feed, or supplement composition, the method comprising:
(a) preparing a cell culture medium, feed, or supplement powder or agglomerated powder;
(b) combining the media powder or agglomerated powder with one or more lubricants, fillers, binders, or combinations thereof; and
(c) producing cell culture medium, feed, or supplement tablets using a tableting apparatus.

13. The method of claim 12, wherein the cell culture medium, feed, or supplement is an agglomerated powder produced by fluid bed agglomeration.

14. The method of claim 12 or 13, wherein the cell culture medium, feed, or supplement powder or agglomerated powder is combined with a lubricant.

15. The method of any one of claims 12-14, wherein the tableted composition comprises 95-99 percent by mass cell culture medium, feed, or supplement powder or agglomerated powder and 1-5 percent of one or more lubricants.

16. The method of any one of claims 12-15, wherein the lubricant comprises magnesium stearate.

17. The method of claim 12-14, wherein the cell culture medium, feed, or supplement powder or agglomerated powder is combined with a lubricant and a disintegrant.

18. The method of any one of claim 12-14 or 17, wherein the tableted composition comprises 95-99 percent by mass cell culture medium, feed, or supplement powder or agglomerated powder; 1-5 percent of one or more lubricants; and 1-5 percent of one or more disintegrants.

19. The method of any one of claim 12-14 or 17-18, wherein the lubricant comprises magnesium stearate and the disintegrant comprises croscarmellose sodium.

20. The method of any one of claims 12-19, wherein the tablet has a mass of 2.0 to 5.0 g.

21. The method of any one of claims 12-20, wherein the tablet has a hardness of 18-22 kp.

22. The method of any one of claims 12-21, wherein the tablet dissolves within 10-30 min in water at 25° C.

23. A tableted cell culture medium, feed, or supplement produced by the method of any one of claims 12-22.

24. The tableted cell culture medium, feed, or supplement of claim 23, wherein the tablet comprises:
a media component comprising:
20-65 percent by mass carbohydrates;
20-40 percent by mass amino acids;
2-10 percent by mass inorganic salts or buffers;
1-5 percent by mass vitamins;
0.01-0.05 percent by mass trace minerals; and
a tableting component comprising:
1-10 percent by mass of a lubricant, filler, binder, or combinations thereof.

25. The tableted cell culture medium, feed, or supplement of claim 24, wherein the tablet comprises 95-99 percent by mass cell culture medium, feed, or supplement; and 1-5 percent of one or more lubricants.

26. The tableted cell culture medium, feed, or supplement of claim 24, wherein the tablet comprises 90-99 percent by mass cell culture medium, feed, or supplement; 1-5 percent by mass of a disintegrant comprising croscarmellose sodium; and 1-5 percent by mass of a lubricant comprising magnesium stearate.

27. The tableted cell culture medium, feed, or supplement of any one of claims 23-26, wherein the tablet has a mass of 2.0 to 5.0 g.

28. The tableted cell culture medium, feed, or supplement of any one of claims 23-27, wherein the tablet has a hardness of 18-22 kp.

29. The tableted cell culture medium, feed, or supplement of any one of claims 23-28, wherein the tablet dissolves within 10-30 min in water at 25° C.

30. A kit comprising a package comprising one or more tablets of the tableted composition of claim 1; a diluent; a receptacle suitable for reconstituting the one or more tablets; and instructions for use.

31. A method for making a cell culture medium, feed, supplement, or buffer from a tableted cell culture medium, feed, supplement, or buffer composition, the method comprising:
(a) combining one or more tablets of cell culture medium, feed, supplement, or buffer with water until dissolved;
(b) optionally, adding any supplements comprising amino acids, antibiotics, sera, or other cell culture media supplements; and
(c) optionally, sterilizing the reconstituted cell culture medium, feeds, supplements, or buffers.

32. The method of claim 31, wherein the sterilization comprises filtration or gamma irradiation.

33. The method of claim 31 or 32, wherein the tablet dissolves within about 10-30 min in water at 25° C.

34. A cell culture medium, feed, supplement, or buffer prepared by the method of any one of claims 31-33.

35. A system comprising a cell and a cell culture medium, feed, supplement, or buffer of claim 34.

36. A method of culturing a cell in a liquid reconstituted from a tableted cell culture medium comprising:
reconstituting a tableted cell culture medium, feed, or supplement in a suitable liquid or buffer; and
culturing the cell in the reconstituted medium under conditions favorable for growth.

37. A method of optimizing the concentration of a cell culture media component, the method comprising:
measuring the concentration of one or more media components in a cell culture;
determining whether the one or more media components are within an acceptable concentration range;
if needed, supplementing the one or more media components in the cell culture with a tableted cell culture media composition.

38. The method of claim 37, wherein the measuring comprises a method selected from HPLC, mass spectrometry, ELISA, or standard curve assay.

39. The method of claim 37, wherein the supplementation comprises adding the tableted media composition direct to the culture or dissolving the tableted media composition in a solvent and then adding the dissolved tableted media composition to the cell culture.

40. Use of a tableted cell culture medium, feed, or supplement for preparing a cell culture medium, feed, or supplement.

41. Use of a tableted cell culture medium, feed, or supplement for culturing cells.

42. The use of claim 41, wherein the tableted cell culture medium, feed, or supplement for culturing is reconstituted and the cell cultured under favorable growth conditions.
